(19) United States
(12) Patent Application Publication
(10) Pub. No.: US 2018/0044637 A1
(43) Pub. Date: Feb. 15, 2018

(54) METHODS FOR GROWING CELLS

(71) Applicant: Children's Hospital Medical Center, Cincinnati, OH (US)

(72) Inventors: Hartmut GEIGER, Cincinnati, OH (US); Sachin KUMAR, Cincinnati, OH (US); Jeffrey D. VASSALLO, Cincinnati, OH (US)

(73) Assignee: Children's Hospital Medical Center, Cincinnati, OH (US)

(21) Appl. No.: 15/673,016

(22) Filed: Aug. 9, 2017

Related U.S. Application Data
(60) Provisional application No. 62/373,015, filed on Aug. 10, 2016.

(51) Int. Cl. C12N 5/0789; C12N 5/0775; C07K 14/535

(52) U.S. Cl. CPC C12N 5/0647; C07K 14/535; C12N 5/0663; C12M 3/02

(57) ABSTRACT

Some embodiments of the invention include methods for growing stem cells comprising growing the stem cells in a cell medium. In certain embodiments, the cell medium has a pH of from about 6.6 to about 7.2. Other embodiments of the invention embodiments include methods for transplanting comprising (a) growing first stem cells in a cell medium having a pH of from about 6.6 to about 7.2, to provide second stem cells and (b) transplanting the second stem cells into a recipient. Other embodiments include the methods where the stem cells that are human stem cells, mouse stem cells, rat stem cells, primate stem cells, or mammalian stem cells. Still other embodiments include growing stem cells in a cell medium having a pH of from about 6.6 to about 7.2 (e.g., from about 6.8 to about 7.0 or about 6.9) that results in modulation of one or more cell properties (e.g., decreased number of cells in the S phase, decreased amount of ROS, etc.) when that property is compared to that of stems cells grown at a pH of about 7.4. Additional embodiments of the invention are also discussed herein.

### CROSS-REFERENCE TO RELATED APPLICATIONS

This application claims the benefit of U.S. Provisional Application No. 62/373,015, filed Aug. 10, 2016, entitled “MEDIA WITH A PH TO EXTEND THE LIFE OF STEM CELLS EX VIVO” which is herein incorporated by reference in its entirety.

### BACKGROUND

Stem cells have numerous uses, such as for research and for treatment of a variety of diseases. However, numerous factors (e.g., growth conditions, oxygen levels, cell age etc.) can contribute to the effectiveness of stems cells and alter their propensity for differentiation. Additionally, the properties of the stem cell can have an impact on the ability for their use in a variety of manner, such as, for example, transplantation. Accordingly, some embodiments of the present invention include methods for growing stem cells in cell medium that address one or more of the issues. Additional embodiments of the invention are also discussed herein.

### SUMMARY

Some embodiments of the present invention include methods for growing first stem cells comprising growing the first stem cells in a cell medium having a pH of from about 6.6 to about 7.2, and recovering second stem cells. In certain embodiments, the pH of the cell medium is from about 6.7 to about 7.1, or is from about 6.8 to about 7.0, or is about 6.9. In still other embodiments, the first stem cells are grown ex vivo. In yet other embodiments, the first stem cells are human stem cells, mouse stem cells, rat stem cells, primate stem cells, or mammalian stem cells. And in yet other embodiments, the first stem cells are somatic stem cells, tissue-specific stem cells, hematopoietic stem cells (HSC), stem cells that express CD34, mammary stem cells, intestinal stem cells, mesenchymal stem cells, endothelial stem cells, neural stem cells, olfactory adult stem cells, neural crest stem cells, or testicular stem cells. In some embodiments, there is an increase in an amount of chimera cells in peripheral blood, when the second stem cells are competitively transplanted into a recipient, when compared to transplanted stem cells that were grown in cell medium of pH about 7.4 (e.g., an increase of at least about 20%). In other embodiments, the second stem cells have a decreased percentage of cells that are in the S phase when compared to stem cells grown in cell medium of pH about 7.4 (e.g., a decrease of at least about 20%). In still other embodiments, second stem cells have a decreased amount of reactive oxygen species compared to stem cells grown in cell medium of pH about 7.4 (e.g., a decrease of at least about 20%). In yet other embodiments, the second stem cells have a decreased level of CD34 expression when compared to stem cells grown in cell medium of pH about 7.4 (e.g., a decrease of at least about 20%). In some embodiments. the second stem cells have a decreased volume of the stem cell when compared to stem cells grown in cell medium of pH about 7.4 (e.g., a decrease of at least about 20%). In certain embodiments, the second stem cells have a decreased amount of H4K16ac in the stem cell when compared to stem cells grown in cell medium of pH about 7.4 (e.g., a decrease of at least about 20%). In other embodiments, the second stem cells have an increased glutathione expression in the stem cell when compared to stem cells grown in cell medium of pH about 7.4 (e.g., an increase of at least about 20%). In some embodiments, the second stem cells have a decreased expression of one or more proteases (e.g., the one or more proteases are cathepsin G, granzyme B, or both) in the stem cell when compared to stem cells grown in cell medium of pH about 7.4 (e.g., a decrease of at least about 20%). In still other embodiments, the first stem cells are taken from a mouse with an age of at least about 2 months, at least about 10 months, or at least about 18 months. In other embodiments, the first stem cells are taken from a human with an age of at least about 10 years, at least about 50 years, or at least about 65 years. In yet other embodiments, recovering comprises one or more of centrifuging, filtering, or washing.

Some embodiments of the invention include methods for transplanting comprising (a) growing first stem cells in a cell medium having a pH of from about 6.6 to about 7.2, to provide second stem cells and (b) transplanting the second stem cells into a recipient. In certain embodiments, the recipient is a mammal, rodent, primate, monkey, human, canine, feline, porcine, mouse, rabbit, or rat. In other embodiments, the transplanting of the second stem cells in the recipient occurs in the bone, bone marrow, blood, skin, brain, or mammary. In yet other embodiments, the pH of the cell medium is from about 6.8 to about 7.0 or is about 6.9. In some embodiments, the first stem cells are grown ex vivo. In certain embodiments, the first stem cells are human stem cells, mouse stem cells, rat stem cells, primate stem cells, or mammalian stem cells, or are somatic stem cells, tissue-specific stem cells, hematopoietic stem cells (HSC), stem cells that express CD34, mammary stem cells, intestinal stem cells, mesenchymal stem cells, endothelial stem cells, neural stem cells, olfactory adult stem cells, neural crest stem cells, or testicular stem cells. In some embodiments, the first stem cells are hematopoietic stem cells (HSC) or stem cells that express CD34. In other embodiments, the method further comprises recovering the second stem cells before step (b).

Other embodiments of the invention are also discussed herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present invention. The invention may be better understood by reference to one or more of these drawings in combination with the description of specific embodiments presented herein.

FIG. 1: Experimental set-up for the measurement of pH in mouse bone marrow. (A) 0.6 mm combination pH probe from Specialty Sensor LLC (USA). (B) Standard pH curve. (C-E) Three-way controlled set-up pH measurement in bone marrow showing a box with grounding loop to avoid interference.

FIG. 2: In vivo analysis of bone marrow (BM) pH. (A) BM pH in young C57Bl/6 mice (2-3 months old) and aged C57Bl/6 mice (22 months-old) measured by using a 0.6 mm combination pH probe, calibrated with 4.00, 7.00 and 10.00 standards with >0.98 R² reading. (B) BM pH measurements after a standard G-CSF regimen treatment for 5 days, commonly used for HSC mobilization. (C) Representative 2-photon spectral analysis of pH sensitive probe, HPTS from 700-950 nm at different pH. (D) Standard curve of HPTS dye against pH by using pH-sensitive 850 nm/pH-neutral 750 nm spectra. (E) 2-photon analysis of pH sensitive probe, HPTS (green) in bone marrow cavity of live mouse, blood vessels were labeled with dextran TRITC (red) and bone surface was identified with second harmonic generation (blue). (F) Representative ¹H NMR overlay analysis of Histidine peak in BM extracellular matrix of young and aged mouse from C57bl/6 and DBA/2 strain. Results are representative of three to four time repeats.

FIG. 3: Calibration of pH sensitive probe HPTS using 2 photon imaging. (A) HPTS (8-hydroxypyrene-1,3,6-trisulphonic acid) protonated and anion forms. (B) HPTS calibration curve setup. (C) Two-photon images at different wavelengths. (D)-(E) Standard Curves.

FIG. 4: Culture media pH calibration. (A) Media calibration procedure. (B) Calibration pH and Culture pH. (C) Before culture and 48 hours post-culture for young and aged cells.

FIG. 5: Effect of pH on Lin− cell growth. A cell suspension containing hematopoietic progenitors was cultivated at different pHs in medium for 48 hours. We then determined the absolute number of progenitor cells (Lin−, c-Kit+ cells, or LK) in the suspension by flow cytometry. The data demonstrate that we have a maximal expansion of these cells at pH 7.4, for young and aged progenitor cells. In second set of analyses, we determined the potential of progenitor cells to form colonies in semi-solid media (colony-forming unit, CFU) after their exposure for 48 hours to different pHs. This assay is performed at pH 7.4 (regular medium). The data show that the most expansion/function occurred at pH of 7.4 (the graph shows relative numbers, and as the overall number of cells declines at a pH higher than 7.4), and the total progenitor number is the highest at 7.4. For aged cells, it is overall a similar picture, with 7.2 to 7.4 being the efficient conditions for an expansion. (A) Effect of pH on Lin− cell number. (B) Effect of pH on LK, LSK, and LT-HSC CFU. (C) Effect of pH on Lin− cell CFU.

FIG. 6: Impact of pH on long term reconstitution of Hematopoietic stem cells (HSCs). (A) Scheme of the experimental set-up for the competitive BM transplants. Young and aged LT-HSC (100 cells) from C57BL/6 mice (Ly5.2) were cultured under pH 7.4 and pH 6.9 settings for 40 h and transplanted with 2×10⁵ cells from BoyJ mice (Ly5.1) under competitive settings. Kinetics of contribution of total donor-derived Ly5.2+ cells in PB after 4-20 weeks in competitive primary (B) and secondary (E) transplants. Contribution of T cells, B cells, and myeloid cells among donor-derived Ly5.2+ cells in PB after 16-20 weeks in competitive primary (C) and secondary (F) transplants. Contribution of total donor-derived Ly5.2+ cells in BM after 16-20 weeks in competitive primary (D) and secondary (G) transplants. Representative FACS dot plots and quantitative and statistical analysis of LT-HSC, ST-HSC, and LMPP distribution among donor derived LSKs in primary (H) and secondary (I) transplanted mice. *p<0.05, **p<0.01, ***p<0.001; columns are means+SE. The experiment was repeated six times with a cohort of three or four recipient mice per group (n=18).

FIG. 7: Effect of pH on LT-HSC engraftment.

FIG. 8: Effect of pH on LT-HSC engraftment—2° transplant BM.

FIG. 9: Limited dilution of LT-HSC engraftment.

FIG. 10: Effect of pH on LT-HSC growth and homing.

FIG. 11: Effect of pH on LT-HSC growth and cell size.

FIG. 12: Low pH decreases HSC cell division rate and cell size. (A) Microscopic analysis of one hundred young and aged fresh HSCs or after culture under pH 7.4 or pH 6.9 condition for 40 h. (B) Quantitative and statistical analysis of accumulative cell number after 40 h culture vs per input cell. (C) Cell cycle analysis of young and aged HSC after culture under pH 7.4 and pH 6.9 conditions for 40 h by using Edu incorporation assay. (D) Bar graph shows percentage of cells in G1, S and G2/M phase of cell cycle under pH 7.4 or pH 6.9 conditions. * **p<0.01, ***p<0.001; columns are means+SE. Results are representative of four to six time repeats.

FIG. 13: Ex vivo low pH mitigates oxidative stress in LT-HSC and regulates CD34 expression. (A) Scheme of the experimental set-up to access effect of pH on LT-HSC ROS. Representative FACS overlay plots for ROS level using DCF-DA in young, aged HSC after culture under pH 7.4 or pH 6.9 conditions. (B) Quantitative and statistical analysis of accumulative intracellular ROS in cultured HSCs under pH 7.4 or pH 6.9 settings. (C) Representative FACS overlay plots for CD34 expression under pH 7.4 or pH 6.9 conditions in young and aged HSCs. (D) Quantitative PCR analysis of Hif-1a target genes pdk1 and vegfa in young and aged HSCs under pH 7.4 or pH 6.9 settings. (E) Ultra-structural analysis of LT-HSCs including vacuolization and mitochondrion alteration under pH 7.4 or pH 6.9 settings. *p<0.05, **p<0.01, ***p<0.001; columns are means+SE. Results are representative of three to four time repeats.

FIG. 14: Effect of pH on Hif-1 targets and mitochondria structure in HSCs. (A) Effect of pH Bnip3 and HIF-1a expression. (B) Mitochondria structure in HSCs via ultrastructural analysis.

FIG. 15: Effect of pH on mitochondrial activity in HSCs.

FIG. 16: Low pH exposure modulates intracellular pH of LT-HSC and regulates epigenetic polarity. (A) Intracellular pH (pHi) of young and aged HSCs under extracellular pH 7.4 or pH 6.9 setting as measured by Snarf-1 staining. (B) Analysis of cell size of young and aged HSCs under pH 7.4 and pH 6.9 conditions using flow cytometric FSC overlay. (C) Area of single HSCs under pH 7.4 and pH 6.9 conditions was monitored using confocal microscopy. (D) Effect of pH on acetylation of H4K16 at global level by FACS analysis, after culture under pH 7.4 or pH 6.9 condition. (E) Representative distribution of H4K16ac (green) and tubulin (red) in young and aged HSC under pH 7.4 or pH 6.9 conditions. Nuclei were stained with DAPI (blue). Bar=5 μm. H4K16ac is also referred to as AcH4K16 in the figures and below. (F) Percentages of cells polarized for H4K16ac in young, aged HSC under pH 7.4 or pH 6.9 conditions. H4K16ac polar distribution was singularly analyzed and scored for each sample cells. The percentage of polarized cells is plotted over the total number of cells scored. *p<0.05, **p<0.01, ***p<0.001; columns are means+SE. Results are representative of four to six time repeats.

FIG. 17: Intracellular pH using Snarf-1 label.

FIG. 18: AcH3K27 does not exhibit polarity.

FIG. 19: RNA sequence analysis identifies pH derived regulators of HSC.

### DETAILED DESCRIPTION

Some embodiments of the invention include methods comprising growing first stem cells in a cell medium having a pH of from about 6.6 to about 7.2, to provide second stem cells. Some embodiments of the invention include methods for transplanting comprising (a) growing first stem cells in a cell medium having a pH of from about 6.6 to about 7.2, to provide second stem cells and (b) transplanting the second stem cells into a recipient. In certain embodiments, the pH of the cell medium can be about 6.6, about 6.65, about 6.7, about 6.75, about 6.8, about 6.85, about 6.9, about 6.95, about 7.0, about 7.05, about 7.1, about 7.15, about 7.2, from about 6.6 to about 7.2, from about 6.7 to about 7.2, from about 6.7 to about 7.1, from about 6.7 to about 7.0, from about 6.8 to about 7.1, or from about 6.8 to about 7.0. The pH of the cell medium can be measured using any suitable method including but not limited to using a 0.6 mm combination pH probe (Specialty Sensor LLC USA) or using a pre-calibrated standard pH Meter (Toledo, USA). The pH of the cell medium can be adjusted using any suitable technique or means including but not limited to one or more of addition of a strong acid (e.g., HCl), addition of a weak acid (e.g., citric acid), additional of a strong base (e.g., NaOH), additional of a weak base (e.g., alanine), additional of one or more buffers (HEPES), additional of macromolecules that increase the pH, or addition of macromolecules that decrease the pH.

In other embodiments, the method further comprises recovering the second stem cells. Recovering can include any suitable method or means to recover the second stem cells, such as but not limited to one or more of purifying, isolating, centrifuging, filtering, or washing (e.g., 1, 2, 3, 4, 5, 6, or 7 times, with the same or different washing solutions) the second stem cells.

In some embodiments, the recipient can be an animal that is the same or different as the origin of first stem cells. In other embodiments, the recipient can be any suitable animal, such as but not limited to mammals, rodents, primates, monkeys, humans, canine, feline, porcine, mice, rabbits, or rats. In yet other embodiments, the recipient can have the second stem cells transplanted in any suitable place in the recipient's body, such as but limited to an organ, bone, bone marrow, blood, lymph node, brain, liver, kidney, testicle, skin, intestine, stomach, or mammary. In certain embodiments, the transplant can be an autologous transplant, an allogeneic transplant, an umbilical cord transplant (e.g., blood), a parent-child transplant, a haplotype mismatched transplant, or a xenotransplant.

In some embodiments, the first stem cells are grown ex vivo (e.g., living cells or tissues taken from an organism and cultured in one or more laboratory devices (e.g., incubators, flasks, Petri dishes, roller bottles, or multi-well plates)). In certain embodiments, the first stem cells are grown under any suitable technique or condition such as but not limited to one or more of using sterile conditions, using controlled temperature in the cell environment (e.g. from about 36° C. to about 37° C.), using controlled carbon dioxide (e.g., from about 5 to about 7% CO₂) in the cell environment (e.g., in the hood or incubator), using controlled humidity in the cell environment (e.g., in the hood or incubator), using a cell culture hood (e.g., class I, class II or class III), using aseptic technique, using adherent culture techniques, using suspension culture techniques, using tissue culture techniques, or any other suitable technique or condition.

The cell medium can be any suitable cell medium, including but not limited to Gibco StemPro-34 SFM (from Thermo Fischer), PromoCell Hematopoietic Progenitor Cell (HPC) Expansion Medium DXF (from PromoCell), Iscove modified Dulbecco medium (IMDM; Cellgro, Catalog #21-020-CV), Stem-Span medium from stem cell technologies, and media from Miltenyi biotec (e.g., a StemMACS media or a Cytomix media). In some embodiments, the cell media is serum-free or the cell media comprises serum. In some embodiments, the growth conditions (e.g., the cell medium) are not identical to (e.g., are not substantially similar to) growth conditions (e.g., the composition of cell nutrient environment) found in vivo or found in the environment from where the first stem cells came or originated. In other embodiments, the cell media is stabilized (e.g., stabilizing the pH) in an incubator without cells; the amount of time for stabilization can be any suitable time including but not limited to for about 1 hr (hour), about 2 hr, about 3 hr, about 4 hr, about 8 hr, about 12 hr, about 16 hr, about 24 hr, about 28 hr, about 32 hr, about 36 hr, about 40 hr, about 44 hr, about 48 hr, about 52 hr, about 56 hr, about 60 hr, about 64 hr, about 68 hr, about 72 hr, about 76 hr, about 80 hr, about 84 hr, about 88 hr, about 92 hr, about 96 hr, about 3.5 days, about 4 days, about 4.5 days, about 5 days, about 5.5 days, about 6 days, from about 1 hr to about 6 days, no more than about 6 days, at least about 1 hr, from about 12 hr to about 4 days, or from about 24 hr to about 3 days.

The first stem cells that can used in the methods described herein (e.g., growing in a cell medium having a pH of about 6.6 to about 7.2) can be any suitable stem cells, such as but not limited to somatic stem cells, tissue-specific stem cells, hematopoietic stem cells (HSC, also referred to as hemocytoblasts), stem cells that express CD34, mammary stem cells, intestinal stem cells, mesenchymal stem cells, endothelial stem cells, neural stem cells, olfactory adult stem cells, neural crest stem cells, or testicular stem cells. The first stem cells (e.g., HSCs) can come from any suitable animal, such as but not limited to mammals, rodents, primates, monkeys, humans, canine, feline, porcine, mice, rabbits, or rats. In some instances, the first stem cells (e.g., HSCs) can be any suitable stem cells, such as but not limited to primary stem cells, secondary stem cells, cells from an animal (e.g., within any suitable time such as but not limited to within about 1 hour, within about 2 hours, within about 4 hours, within about 8 hours, within about 12 hours, within about 24 hours, or within about 48 hours), or cells thawed from a freezer (e.g., where the thawing took place within any suitable time such as but not limited to within about 1 hour, within about 2 hours, within about 4 hours, within about 8 hours, within about 12 hours, within about 24 hours, or within about 48 hours).

In some embodiments, the first stem cells (e.g., HSCs) are taken (e.g., harvested) from an animal (e.g., a human or a mouse) with an age of about 0.1 mo. (month), about 1 mo., about 2 mo., about 3 mo., about 4 mo., about 5 mo., about 6 mo., about 7 mo., about 8 mo., about 9 mo., about 10 mo., about 11 mo., about 12 mo., about 13 mo., about 14 mo., about 15 mo., about 16 mo., about 17 mo., about 18 mo., about 19 mo., about 20 mo., about 21 mo., about 22 mo., about 23 mo., about 24 mo., about 25 mo., about 26 mo., about 27 mo., about 28 mo., about 29 mo., about 30 mo., about 31 mo., about 32 mo., about 33 mo., about 34 mo., about 35 mo., about 36 mo., about 1 yr., about 1.5 yr., about 2 yr., about 2.5 yr, about 3 yr., about 3.5 year, about 4 yr., about 4.5 yr., about 5 yr., about 7.5 yr., about 10 yr., about 15 yr., about 20 yr., about 25 yr., about 30 yr., about 35 yr., about 40 yr., about 45 yr., about 50 yr., about 55 yr., about 60 yr., about 65 yr., about 70 yr., about 75 yr., about 80 yr., about 85 yr., about 90 yr., about 95 yr., about 100 yr., about 150 yr., no more than about 150 yr., no more than about 100 yr., no more than about 50 yr., no more than about 20 yr., no more than about 5 yr., no more than about 20 mo., no more than about 18 mo., no more than about 5 mo., no more than about 4 mo., no more than about 1 mo., at least about 0.1 mo., at least about 1 mo., at least about 2 mo., at least about 4 mo., at least about 5 mo., at least about 10 mo., at least about 15 mo., at least about 18 mo., at least about 2 yr., at least about 10 yr., at least about 20 yr., at least about 35 yr., at least about 50 yr., at least about 65 yrs., at least about 75 yr., at least about 90 yr., from about 0.1 mo. to about 150 yr., from about 1 mo. about 150 yr., from about 5 mo. to about 100 yr., from about 5 mo. to about 50 yr., from about 5 mo. to about 10 yr., from about 5 mo. to about 24 mo., from about 10 mo. to about 100 yr., from about 10 mo. to about 50 yr., from about 10 mo. to about 10 yr., from about 10 mo. to about 24 mo., from about 15 mo. to about 100 yr., from about 15 mo. to about 50 yr., from about 15 mo. to about 10 yr., from about 15 mo. to about 24 mo., from about 10 yr. to about 100 yr., from about 10 yr. to about 50 yr., from about 20 yr. to about 100 yr., from about 20 yr. to about 50 yr., from about 30 yr. to about 100 yr., from about 30 yr. to about 50 yr., from about 50 yr. to about 100 yr., or from about 75 yr. to about 100 yr.

The first stem cells (e.g., HSCs) can be grown in the cell medium for any suitable amount of time including but not limited to for about 1 hr (hour), about 2 hr, about 3 hr, about 4 hr, about 8 hr, about 12 hr, about 16 hr, about 24 hr, about 28 hr, about 32 hr, about 36 hr, about 40 hr, about 44 hr, about 48 hr, about 52 hr, about 56 hr, about 60 hr, about 64 hr, about 68 hr, about 72 hr, about 76 hr, about 80 hr, about 84 hr, about 88 hr, about 92 hr, about 96 hr, about 3.5 days, about 4 days, about 4.5 days, about 5 days, about 5.5 days, about 6 days, about 7 days, about 8 days, about 9 days, about 10 days, about 11 days, about 12 days, about 13 days, about 14 days, about 2.5 weeks, about 3 weeks, about 3.5 weeks, about 4 weeks, about 1.5 months, about 2 months, about 2.5 months, about 3 months, about 4 months, about 5 months, about 6 months, from about 1 hr to about 6 months, no more than about 6 months, at least about 1 hr, from about 12 hr to about 4 months, or from about 24 hr to about 3 months. In some embodiments, the cell medium is refreshed (e.g., replaced with fresh cell medium of the same type) one or more times during the method (e.g., 1, 2, 3, 4, 5, 6, 7, 8, 9 10, 15, 20, or 25 times); the cell medium can be refreshed at any suitable time from the start of the method or from the previous cell medium refreshing, and the time between any cell medium refreshing can be the same or different as the time between other cell medium refreshings. The time between cell medium refreshing can be any suitable time including but not limited to a one or a combination of about 1 hr (hour), about 2 hr, about 3 hr, about 4 hr, about 8 hr, about 12 hr, about 16 hr, about 24 hr, about 28 hr, about 32 hr, about 36 hr, about 40 hr, about 44 hr, about 48 hr, about 52 hr, about 56 hr, about 60 hr, about 64 hr, about 68 hr, about 72 hr, about 76 hr, about 80 hr, about 84 hr, about 88 hr, about 92 hr, about 96 hr, about 3.5 days, about 4 days, about 4.5 days, about 5 days, about 5.5 days, about 6 days, about 7 days, about 8 days, about 9 days, about 10 days, about 11 days, about 12 days, about 13 days, or about 14 days.

In some embodiments, growing first stem cells in a cell medium having a pH of from about 6.6 to about 7.2 (e.g., from about 6.8 to about 7.0 or about 6.9) can result in modulation of one or more cell properties of second stem cells when that property is compared to that of stems cells grown at a pH of about 7.4 (e.g., in the same or similar cell medium where the only difference or only substantive difference (a substantive difference is one where the given property is changed by no more than about 1%, no more than about 5%, no more than about 10%, or no more than about 15%) in the cell medium is that it has a pH of about 7.4). Modulation can be an increase or a decrease. In some embodiments, one or more of the following cell properties of the second stem cell can, in some instances, be modulated (e.g., as compared to cells grown at a pH about 7.4), but cell properties that can be modulated are not limited to those listed here: (a) an increase in the amount of chimera cells in peripheral blood of a recipient, after second stem cells were competitively transplanted (e.g., a bone marrow transplant, a primary transplant, or a secondary transplant) into the recipient (e.g., a lethally irradiated recipient), where the percentage increase in the amount of chimera cells in peripheral blood can be, for example, about 20%, about 30%, about 40%, about 50%, about 100%, about 120%, about 150%, about 200%, about 250%, about 300%, about 400%, about 500%, about 600%, about 700%, at least about 20%, at least about 50%, or at least about 100%; (b) a decrease in the percentage of second stem cells that are in the S phase of the cell division cycle (i.e., compared to stem cells grown at pH about 7.4), where the decrease can be, for example, about 20%, about 30%, about 40%, about 50%, about 100%, about 120%, about 150%, about 200%, about 250%, about 300%, about 400%, about 500%, about 600%, about 700%, at least about 20%, at least about 50%, or at least about 100%; (c) a decrease in the amount of reactive oxygen species (ROS) (e.g., measured using DCF fluorescence) in the second stem cells (i.e., compared to stem cells grown at pH about 7.4), where the percentage decrease in the amount of ROS can be, for example, about 20%, about 30%, about 40%, about 50%, about 100%, about 120%, about 150%, about 200%, about 250%, about 300%, about 400%, about 500%, about 600%, about 700%, at least about 20%, at least about 50%, or at least about 100%; (d) a decrease in the level of CD34 expression (e.g., in mice) in the second stem cells (i.e., compared to stem cells grown at pH about 7.4), where the percentage decrease in level of CD34 expression can be, for example, about 20%, about 30%, about 40%, about 50%, about 100%, about 120%, about 150%, about 200%, about 250%, about 300%, about 400%, about 500%, about 600%, about 700%, at least about 20%, at least about 50%, or at least about 100%; (e) an increase in stabilization of post-translational Hif1a (hypoxia-inducible factor 1-alpha) in the second stem cells (i.e., compared to stem cells grown at pH about 7.4), such as for example, a lack of change in Hif1a expression (e.g., less than about 70% change, less than about 50% change, less than about 20% change, less than about 10% change, less than about 5% change, less than about 2% change, less than about 1% change, or less than about 0.1% change); (f) a decrease in mitochondrial stress of the second stem cells (i.e., compared to stem cells grown at pH about 7.4), as for example indicated by less elongated mitochondria (i.e., compared to stem cells grown at pH about 7.4) or by a decrease vacuolization (i.e., compared to stem cells grown at pH about 7.4), where the percentage decrease in vacuolization (either in number of vacuoles or in volume occupied by vacuoles) can be, for example, about 20%, about 30%, about 40%, about 50%, about 100%, about 120%, about 150%, about 200%, about 250%, about 300%, about 400%, about 500%, about 600%, about 700%, at least about 20%, at least about 50%, or at least about 100%; (g) a decrease in the intracellular pH (pHi) (e.g., measured via Snarf-1) of the second stem cells (i.e., compared to stem cells grown at pH about 7.4), where the percentage decrease can be, for example, about 20%, about 30%, about 40%, about 50%, about 100%, about 120%, about 150%, about 200%, about 250%, about 300%, about 400%, about 500%, about 600%, about 700%, at least about 20%, at least about 50%, or at least about 100%; (h) a decrease in the volume of the second stem cells (e.g., the mean or the median as, for example, measured by flow cytometry and/or confocal microscopy) (i.e., compared to stem cells grown at pH about 7.4), where the percentage decrease can be, for example, about 20%, about 30%, about 40%, about 50%, about 100%, about 120%, about 150%, about 200%, about 250%, about 300%, about 400%, about 500%, about 600%, about 700%, at least about 20%, at least about 50%, or at least about 100%; (i) a decrease in the amount of H4K16ac (acetylation at histone H4 on lysine 12) in the second stem cells (e.g., using FACS analysis) (i.e., compared to stem cells grown at pH about 7.4), where the percentage decrease can be, for example, about 20%, about 30%, about 40%, about 50%, about 100%, about 120%, about 150%, about 200%, about 250%, about 300%, about 400%, about 500%, about 600%, about 700%, at least about 20%, at least about 50%, or at least about 100%; (j) an increase in the glutathione expression in the second stem cells (i.e., compared to stem cells grown at pH about 7.4), where the percentage increase can be, for example, about 20%, about 30%, about 40%, about 50%, about 100%, about 120%, about 150%, about 200%, about 250%, about 300%, about 400%, about 500%, about 600%, about 700%, at least about 20%, at least about 50%, or at least about 100%; (k) an increase in quiescence and/or in self-renewal (e.g., via a CBFA2T3-related mechanism) in the second stem cells (i.e., compared to stem cells grown at pH about 7.4); or (l) a decrease in expression of one or more proteases (e.g., one or more of cathepsin G or granzyme B) in the second stem cells (i.e., compared to stem cells grown at pH about 7.4), where the where the percentage decrease for one or more proteases can be the same or different and can be, for example, about 20%, about 30%, about 40%, about 50%, about 100%, about 120%, about 150%, about 200%, about 250%, about 300%, about 400%, about 500%, about 600%, about 700%, at least about 20%, at least about 50%, or at least about 100%.

In certain embodiments, the methods disclosed herein can result in second stem cells that have elevated contributions to the hematopoietic system upon transplantation into recipients. In yet other embodiments, the methods disclosed herein can result in second stem cells that have enhanced long-term contributions to the hematopoietic system upon transplantation (e.g., via transplantation into secondary and tertiary recipients). In still other embodiments, the methods disclosed herein can result in a more healthy differentiation pattern upon transplantation of second stem cells (e.g., when aged HSCs are transplanted; aged can be, for example, about 18 months or older for mice, or about 60 years old or older for humans); “more healthy” can include but is not limited to less monocytes/macrophages or more lymphoid cells. In additional embodiments, the methods disclosed herein can result in an enhanced time to neutrophil recovery after transplantation of second stem cells.

In some embodiments, the methods described herein can be part of any suitable method or procedure, such as but not limited to ex vivo manipulation of stem cells (e.g., HSCs), collecting stem cells (e.g., HSCs), collecting and freezing stem cells (e.g., HSCs), thawing stem cells (e.g., HSCs), maintaining ex vivo stems cells (e.g., HSCs) as part of genetically modifying stem cells (e.g., HSCs), transplanting stem cells (e.g., HSCs), transplanting stem cells (e.g., HSCs) where the stem cells (e.g., HSCs) were previously frozen, transplanting stem cells (e.g., HSCs) where the stem cells (e.g., HSCs) were not previously frozen, and research methods for studying stem cells (e.g., HSCs).

The presently-disclosed subject matter is further illustrated by the following specific but non-limiting examples. The following examples may include compilations of data that are representative of data gathered at various times during the course of development and experimentation related to the present invention.

### EXAMPLES

### pH in Bone Marrow (BM) is Low and in Flux Upon Aging

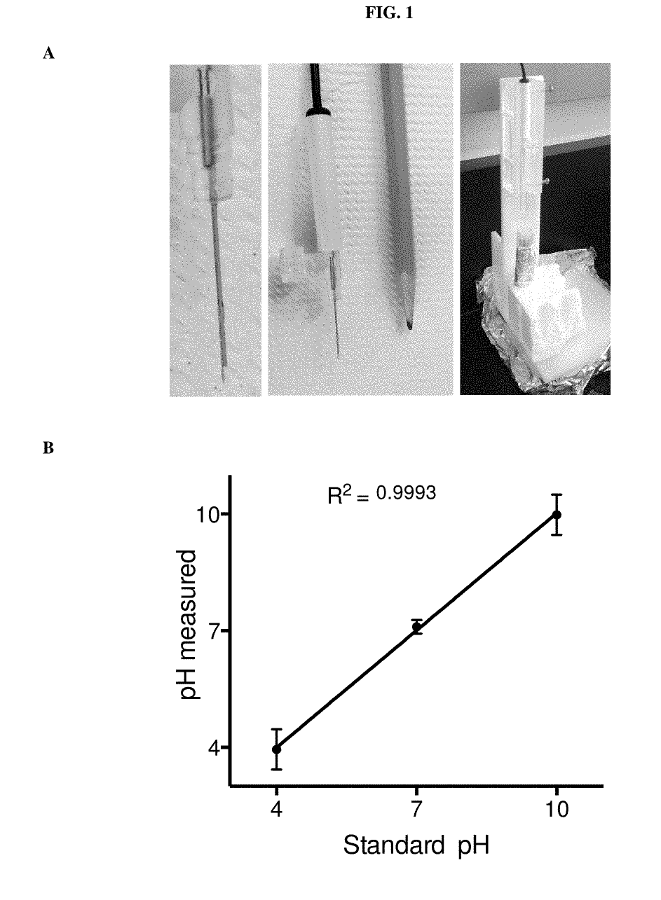

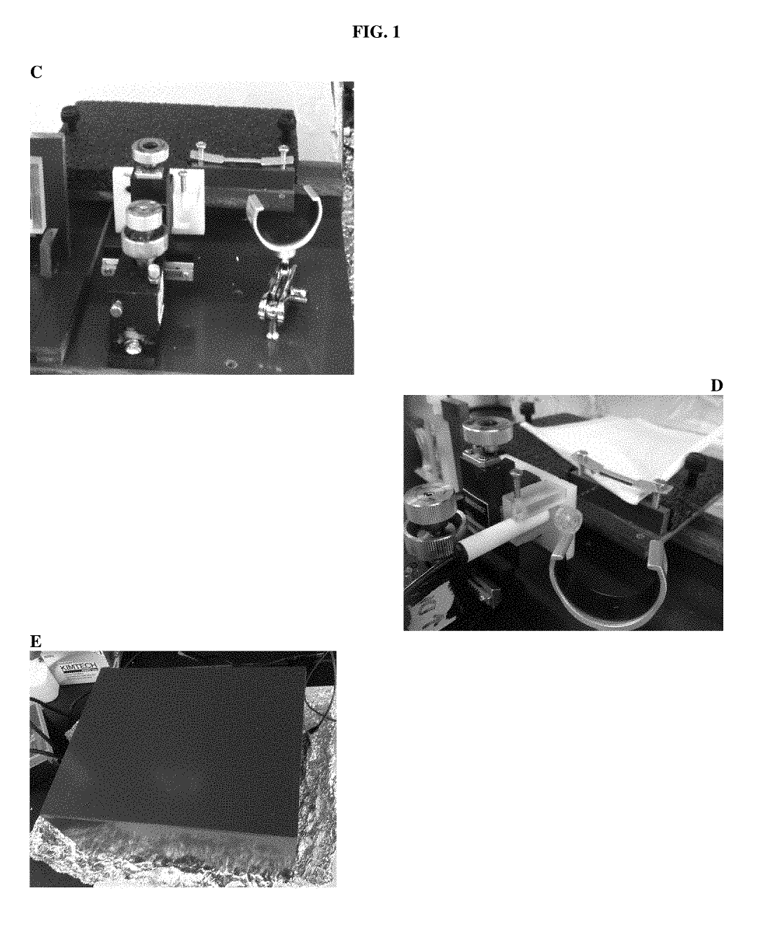

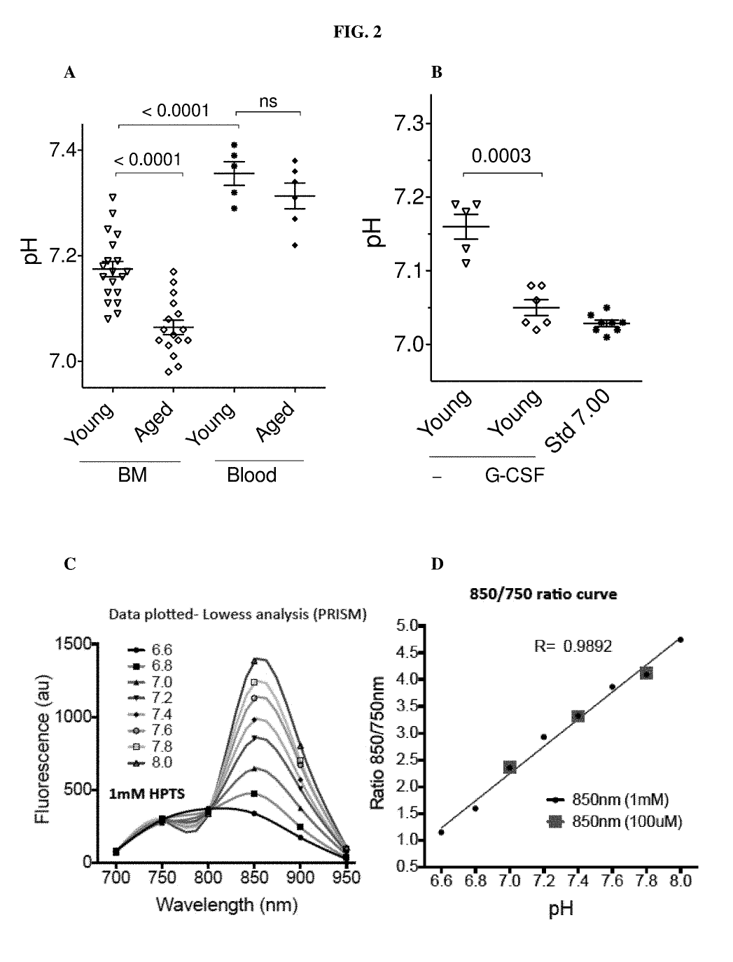

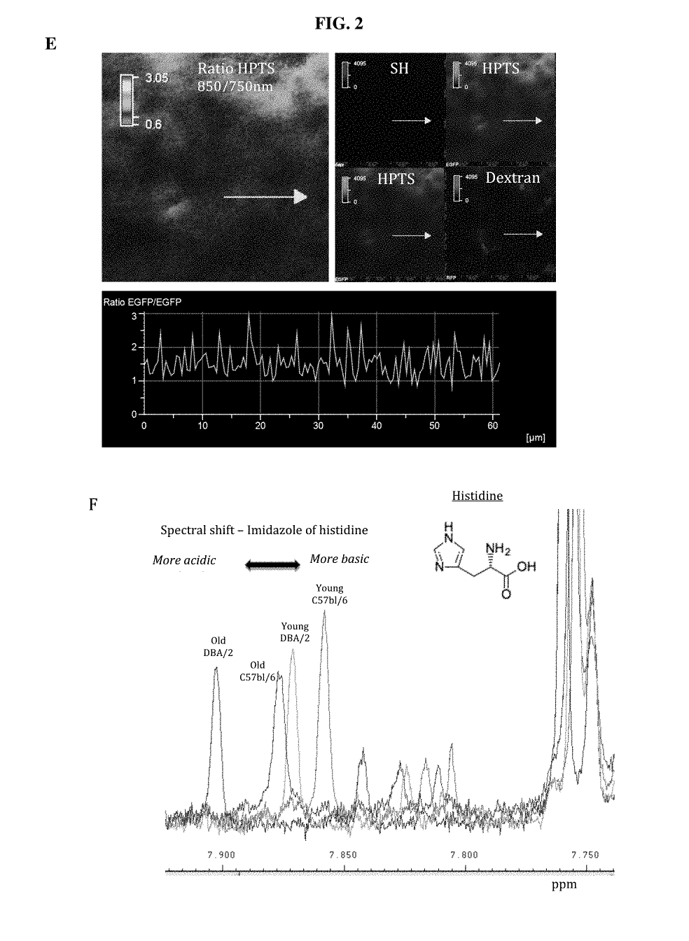

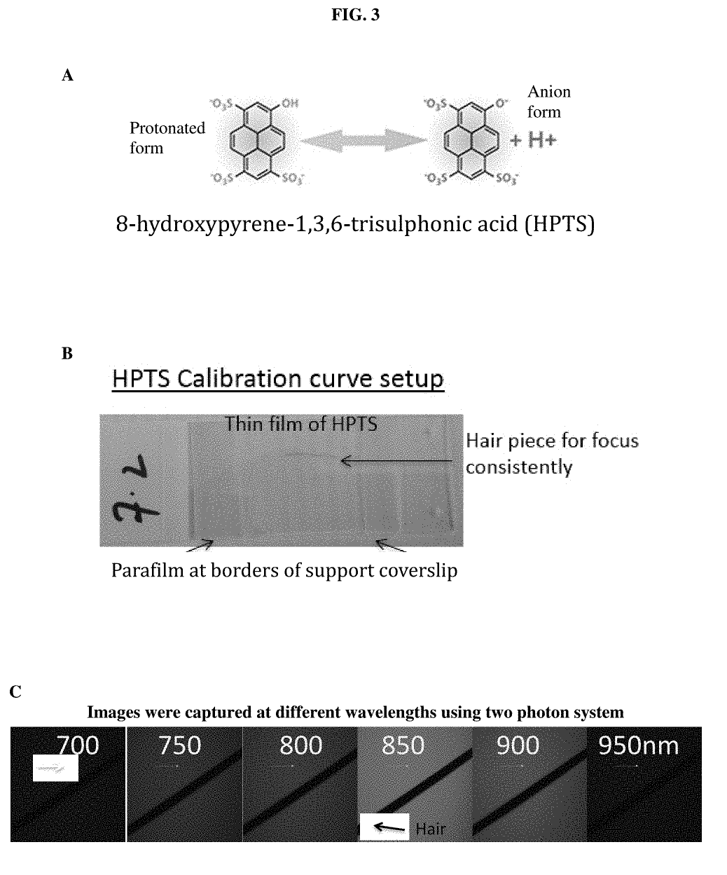

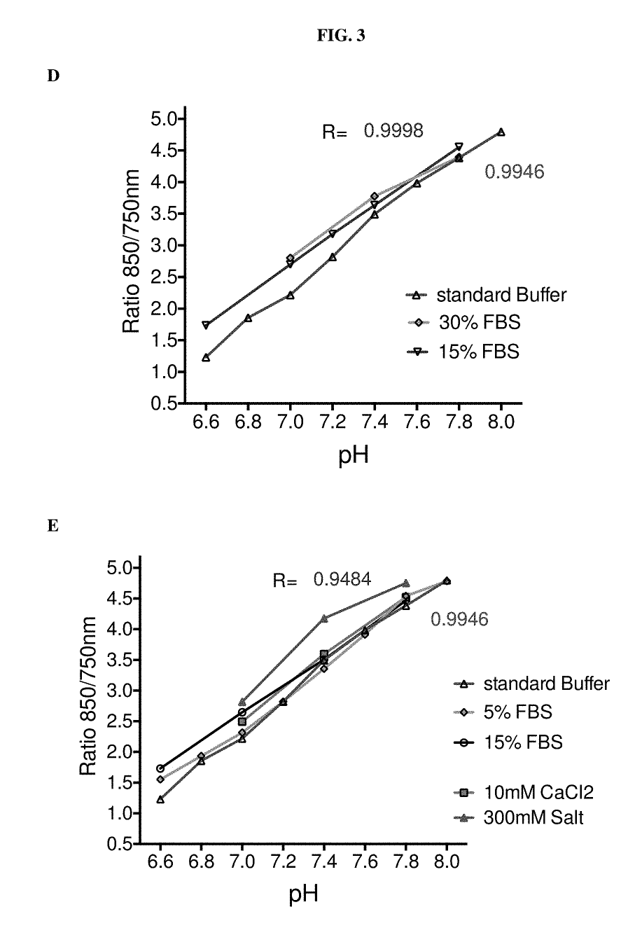

We first set out to determine pH levels within the BM of both young (i.e., 2-4 months old) mice and aged (i.e., >18 months old) mice using multiple complimentary approaches. We initially utilized a small 0.6 mm combined glass pH electrode to measure pH directly in the bone cavity in vivo (FIGS. 1A-1E). pH in bone marrow (BM) of young mice was significantly lower (pH 7.19±0.01) than that of peripheral blood (about 7.4, as expected). Aged mice showed even a lower BM pH (pH 7.05±0.01), (FIG. 2A) with still a pH of about 7.35 in blood. Granulocyte-colony stimulating factor (G-CSF) given to mice results in mobilization of hematopoietic stem cells (HSCs) out of the bone marrow. The pH within bone marrow of young animals that received G-CSF for mobilization was decreased to about pH 7.05 (FIG. 2B), also validating our method of pH determination within the BM (FIG. 2A). Together these data suggest active regulation of extracellular pH upon G-CSF administration, aging (FIG. 2B) and irradiation (also lowered pH, data not shown). We next tested whether pH within BM is uniform, or, whether, as suggested by others, there are indeed pH pockets (local fluctuations of pH) within BM. To test this we utilized live multi-photon microscopy to detect BM pH in situ by using an established cell impermeable pH sensitive ratio-metric probe, 8-Hydroxypyrene-1,3,6-Trisulfonic Acid (HPTS) and performed additional standardization experiments (FIG. 1 C, FIG. D, and FIG. 3)—see, for example, RAY et al., “Two-photon fluorescence imaging super-enhanced by multishell nanophotonic particles, with application to subcellular pH” (2012) Small, Vol. 8, pp. 2213-2221, which is herein incorporated by reference in its entirety. Changes in pH are represented by a change in the ratio of fluorescence for the pH insensitive wavelength of the dye (750 nm) vs. the pH sensitive wavelength of the dye (850 nm), FIG. 2C. We detected a differential pH pattern throughout the BM cavity, as different pH values were observed within the BM (FIG. 2E), ranging from 6.6 up to about 7.2. Thus, and without being bound by theory, stem cells might reside inside distinct pH pockets in vivo; a hypothesis that will need to await further experimental confirmation, as currently technology does not likely permit both stem cell identification and pH measurements directly at that scale in vivo. Using a metabolomics approach on the extracellular fluid fraction of bone marrow, we observed a spectral shift in imidazole at histidine in aged compared to young BM in C57BL/6 mice. Such a shift occurs at a lower pH and thus further verifies a decrease in pH in BM upon aging (FIG. 2F). BM fluid from young and aged DBA/2 mice also exhibit a similar shift (FIG. 2F), indicating that an aging-associated drop in pH is not restricted to C57BL/6 mice.

### Extracellular pH Regulates HSCs Function

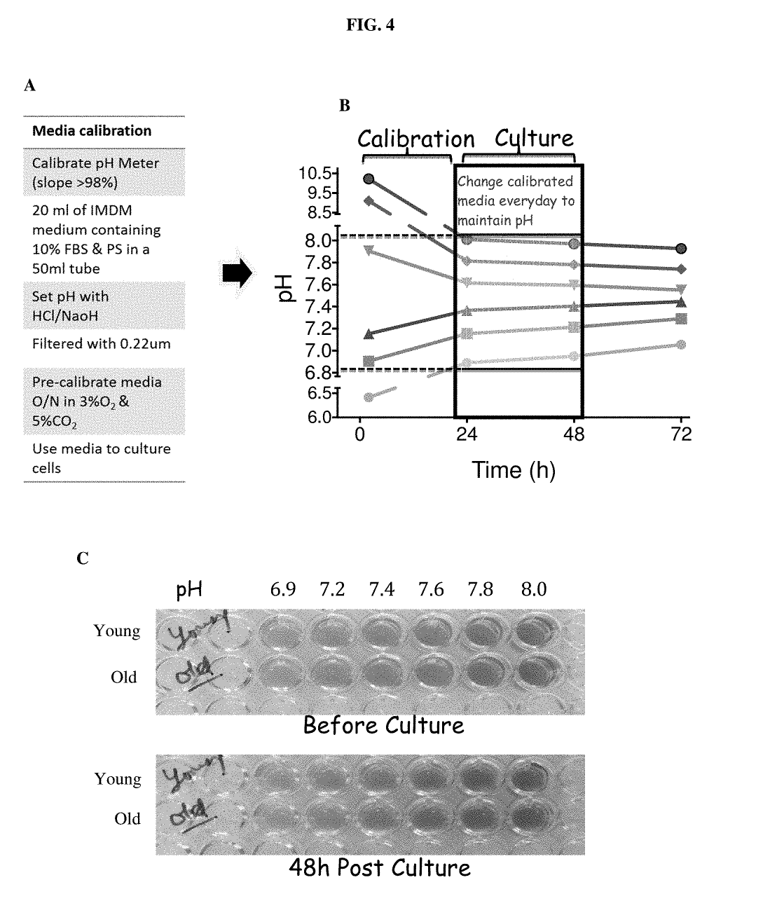

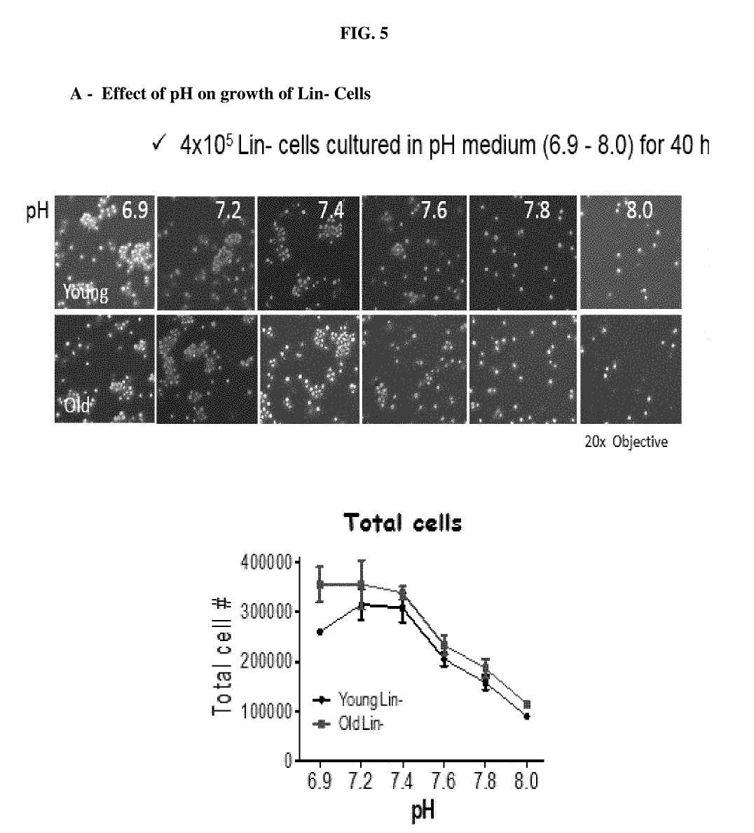

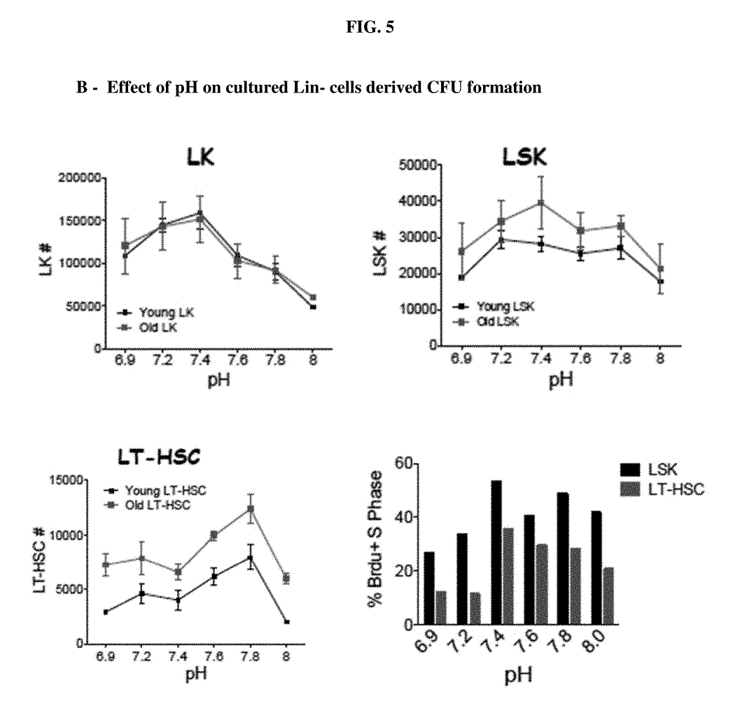

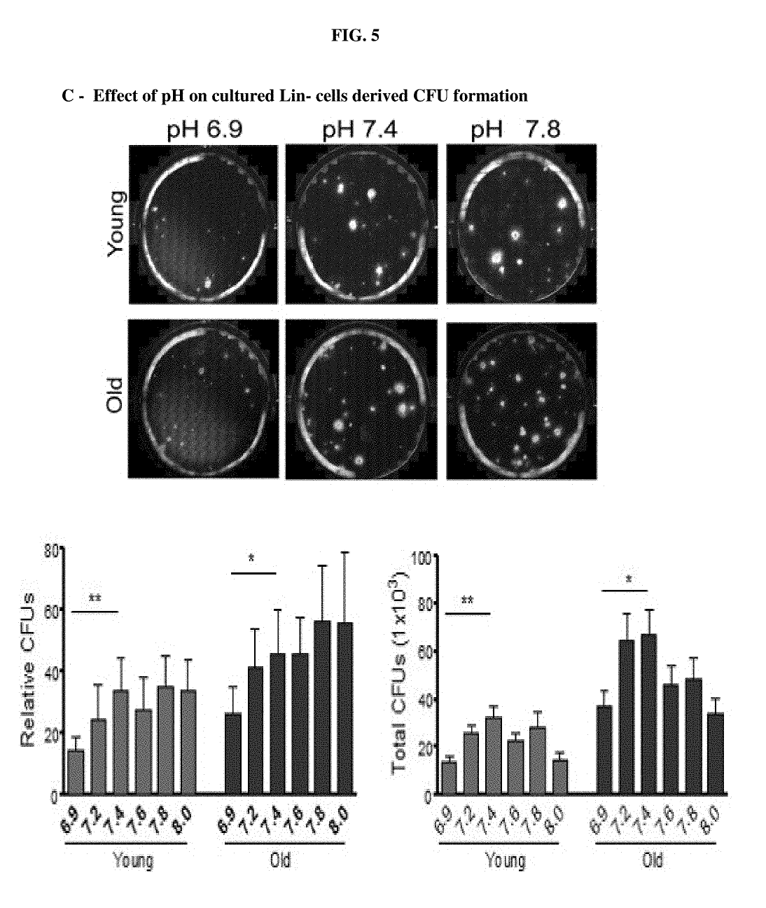

We incubated cells enriched for hematopoietic stem cells (HSCs) and progenitor cells (LIN-cells) from both young (i.e., 2-4 months old) mice and aged (i.e., >18 months old) mice for 48 hours in a pH ranging from 6.9 to 8.0 (FIG. 4) and subsequently determined hematopoietic progenitor activity in the cell population with a colony-forming cell assay. Cells kept at a pH of 6.9 resulted in a decrease in the CFU frequency compared to cultivation at pH 7.4 (FIG. 5). These data imply that primitive hematopoietic stem cells respond differently at different pHs.

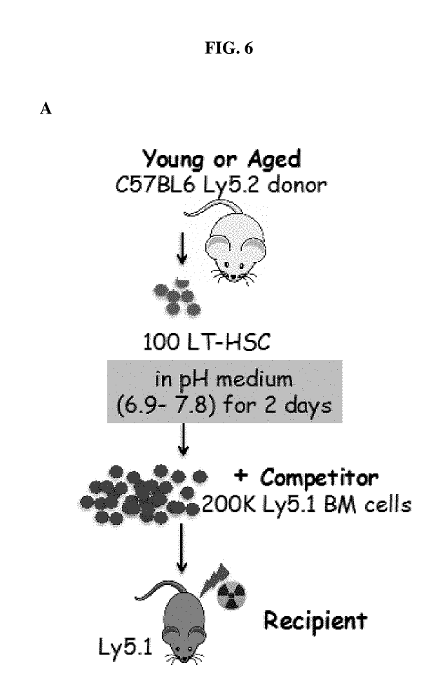

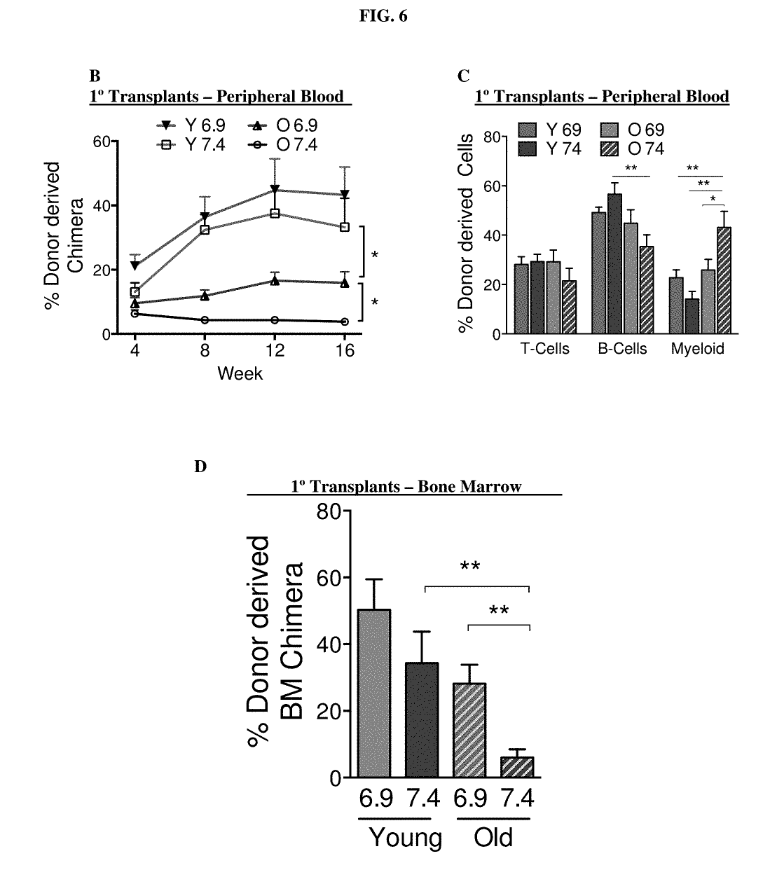

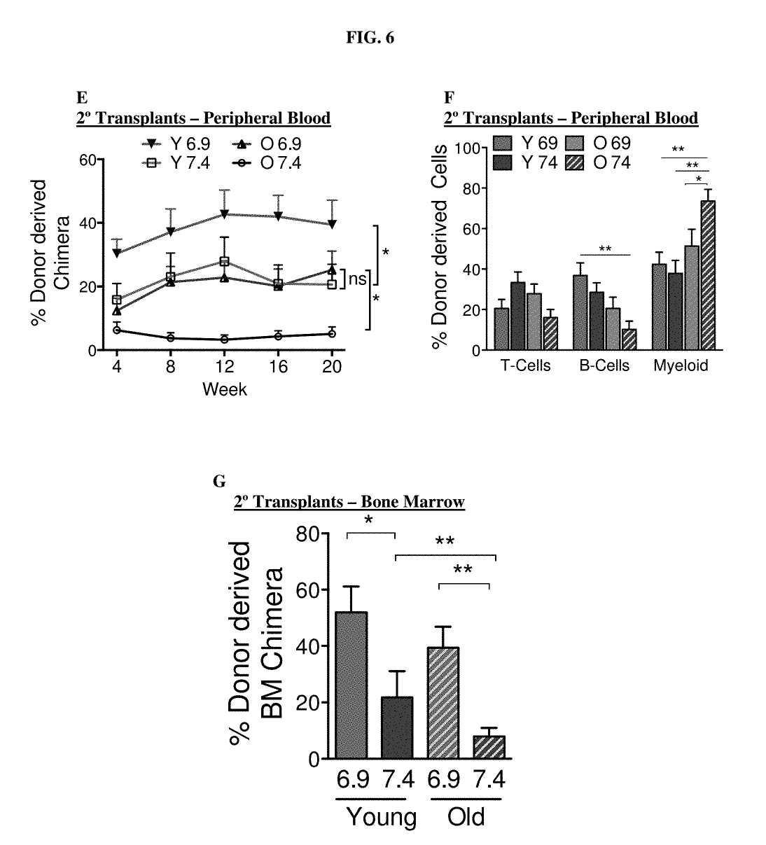

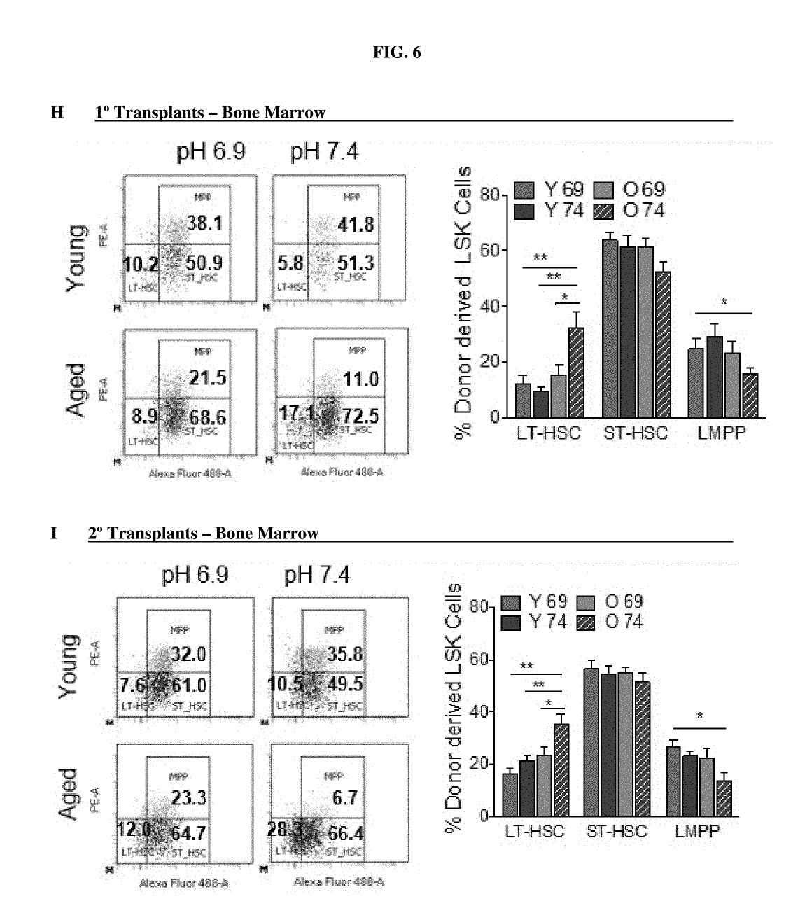

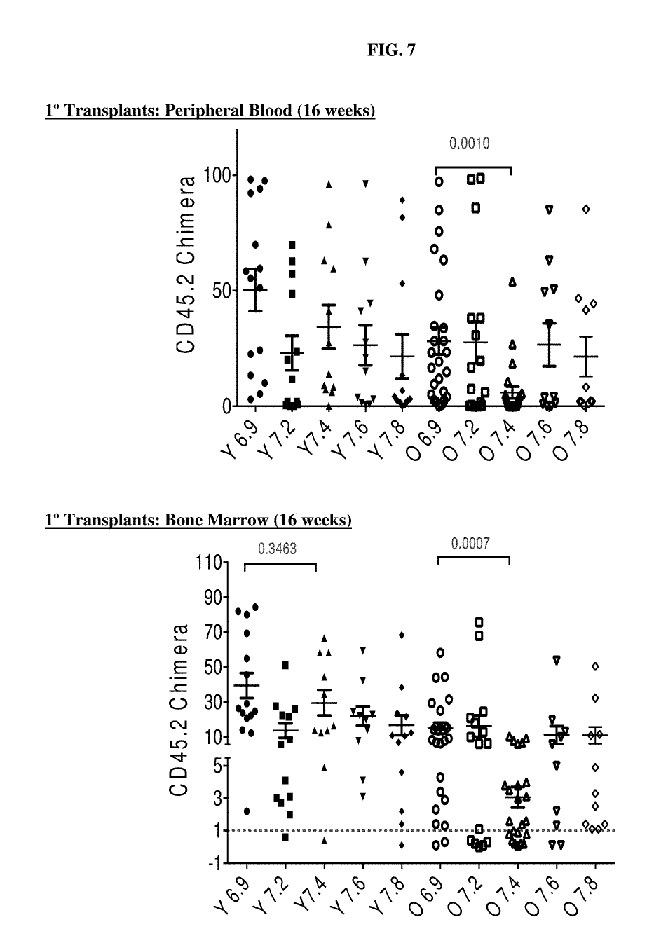

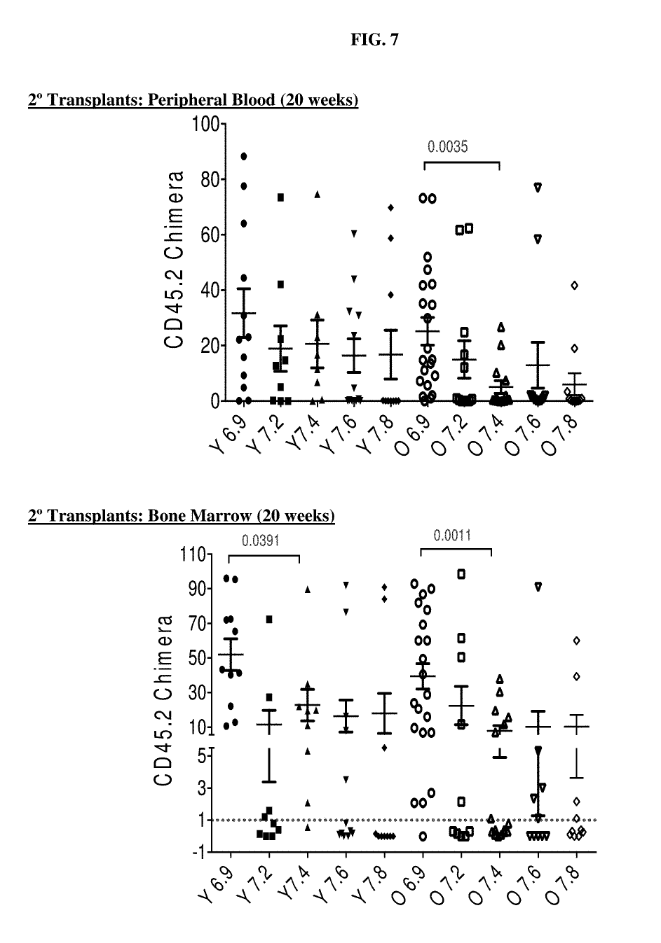

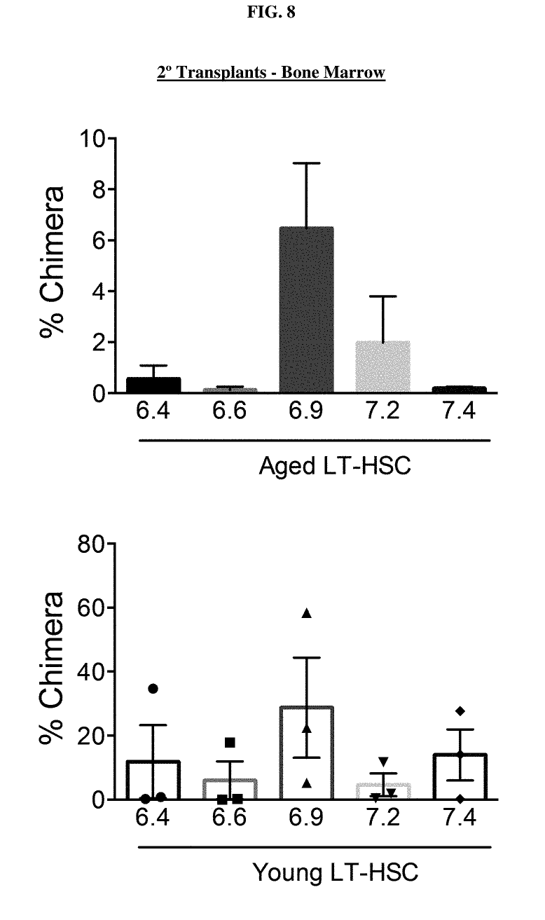

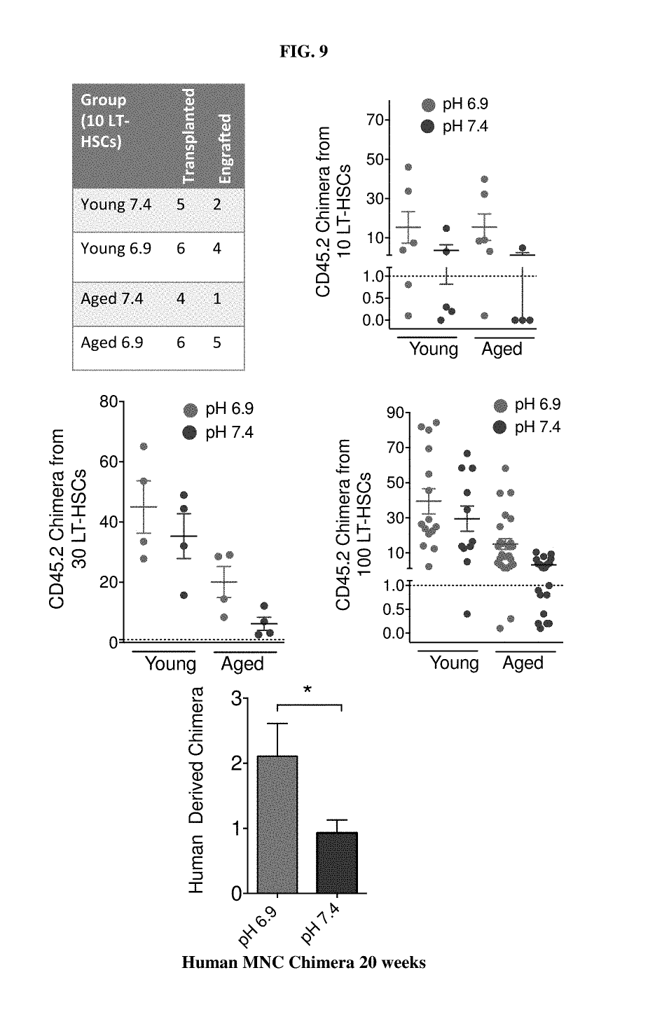

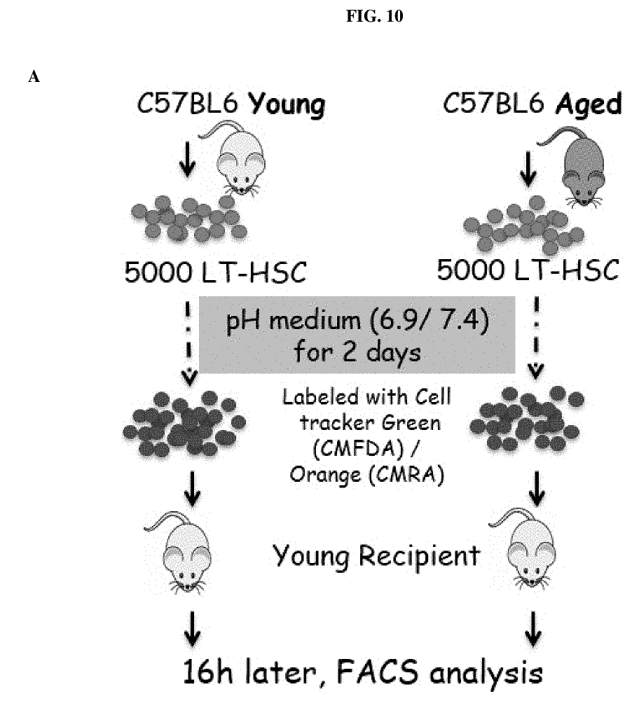

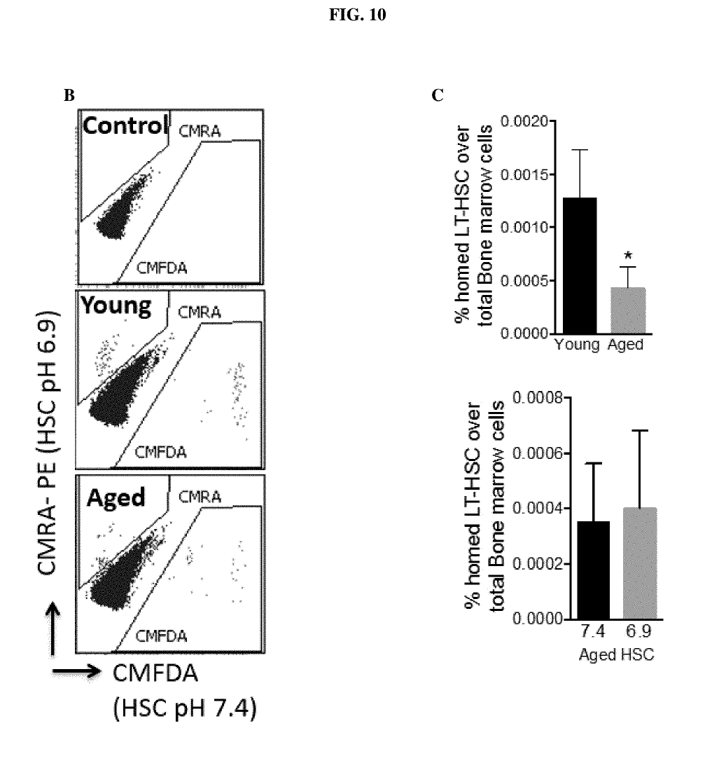

To determine some effects of changes in pH on HSC function, isolated HSCs (Lin⁻Sca-1⁺c-Kit⁺CD34⁻Flt3⁻ cells) from BM were exposed to conditions ranging from pH 6.4-7.8 for 48 hours and subsequently competitively transplanted into the bone marrow of lethally irradiated recipients (FIG. 6A, FIG. 7, and FIG. 8), to determine stem cell function. Aged HSCs, compared to young HSCs, are impaired, as shown in the serial (primary and secondary) repopulation assay. For example, an overall lower chimerism occurred in animals transplanted with aged HSCs as compared to younger HSCs. Also, the aged HSCs presented with elevated contribution to the myeloid lineage at the expense of B-cell contribution. Both young and aged HSCs exposed to a pH 6.9 though, compared to any other tested pH condition (FIG. 7 and FIG. 8) presented with a higher level of chimerism in peripheral blood in primary (FIG. 6B) as well as secondary recipients (FIG. 6E) with a relative increase in the repopulation ability of especially aged HSCs exposed to a pH 6.9 (FIG. 6D and FIG. 6G). Aged HSCs exposed to a pH of 6.9 even functionally resembled young HSCs at pH 6.9 with respect to the contribution to the B and myeloid cell lineage in PB in primary (FIG. 6C) and secondary recipients (FIG. 6F). Enhanced function of young and aged HSCs exposed to pH 6.9 compared to the pH 7.4 “standard” was further confirmed in limiting dilution transplantation experiments, using only 10 or 30 donor HSCs (FIG. 9). Also, human HSCs, when exposed to a pH of 6.9 instead of 7.4, present with an elevated contribution upon xenotransplantation into NSG mice (FIG. 9). Interestingly, aged HSCs exposed to a pH 6.9 presented with frequencies of B-cells and myeloid cells in blood and overall chimerism in bone marrow similar to young HSCs exposed to a standard pH of 7.4 (FIG. 6A-G). Aged HSCs, exposed to a pH of 6.9, upon transplantation also showed a more youthful lower level of contribution to the pool of LT-HSCs in both primary (FIGS. 6H, 7, and 8) as well as secondary recipients (FIGS. 6I, 7, and 8). Competitive short-term stem cell homing assays revealed that while aged HSCs present with reduced homing, incubation at a pH of 6.9 did not show an apparent difference in homing ability compared to pH 7.4, indicating that changes in pH do not appear to alter stem cell homing (FIG. 10). Together, these results demonstrate that both young and aged HSCs exposed to a pH of 6.9 present with enhanced stem cell function compared to HSCs exposed to a pH of 7.4.

### HSCs Alter Proliferation and Growth Upon pH Changes

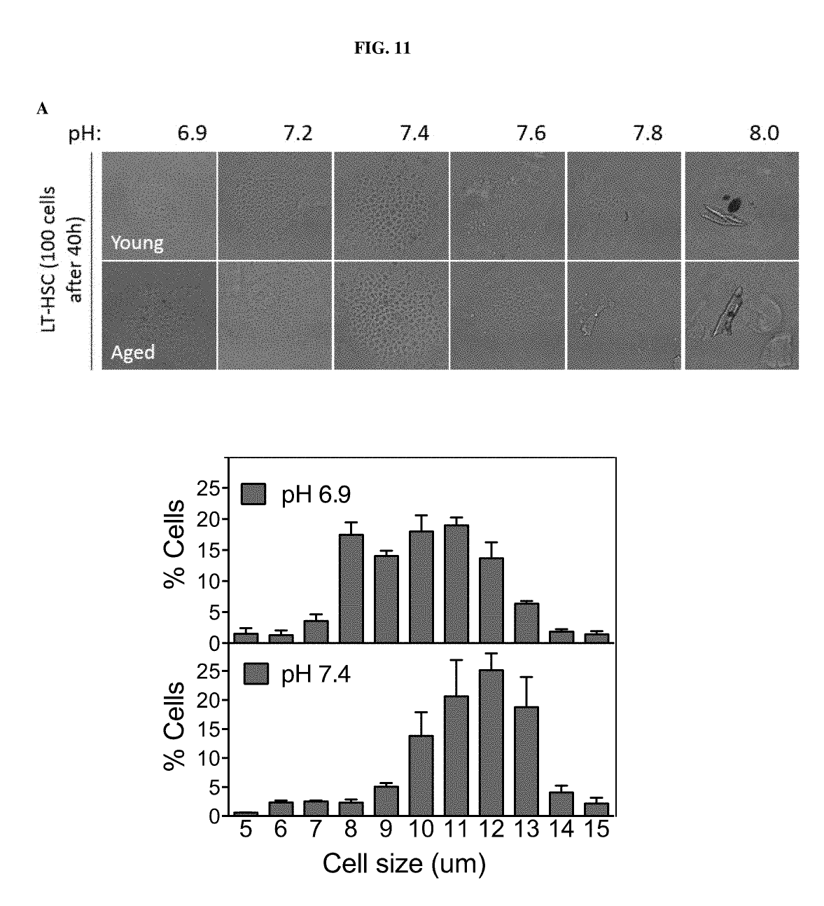

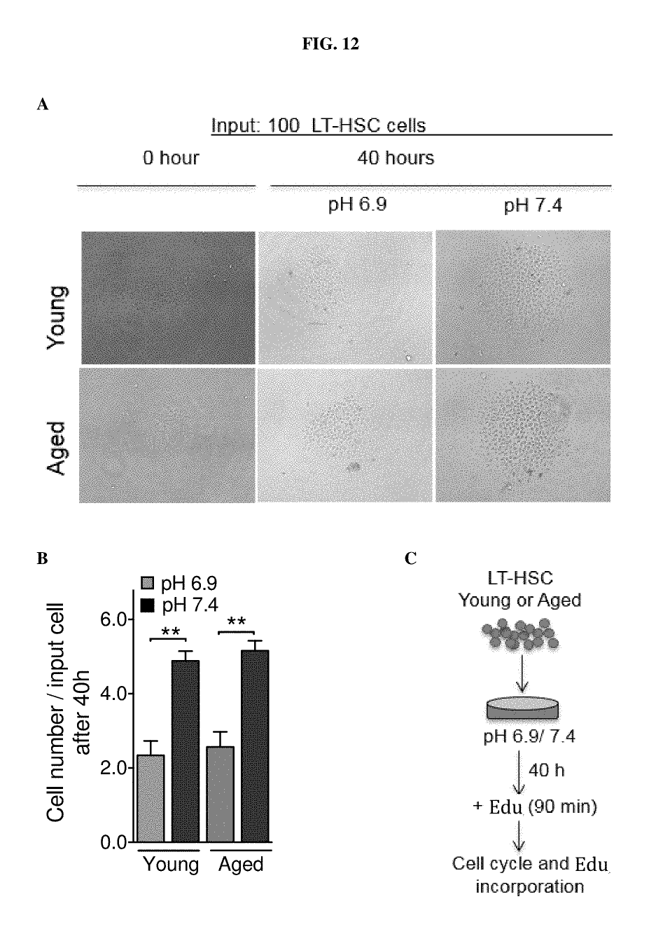

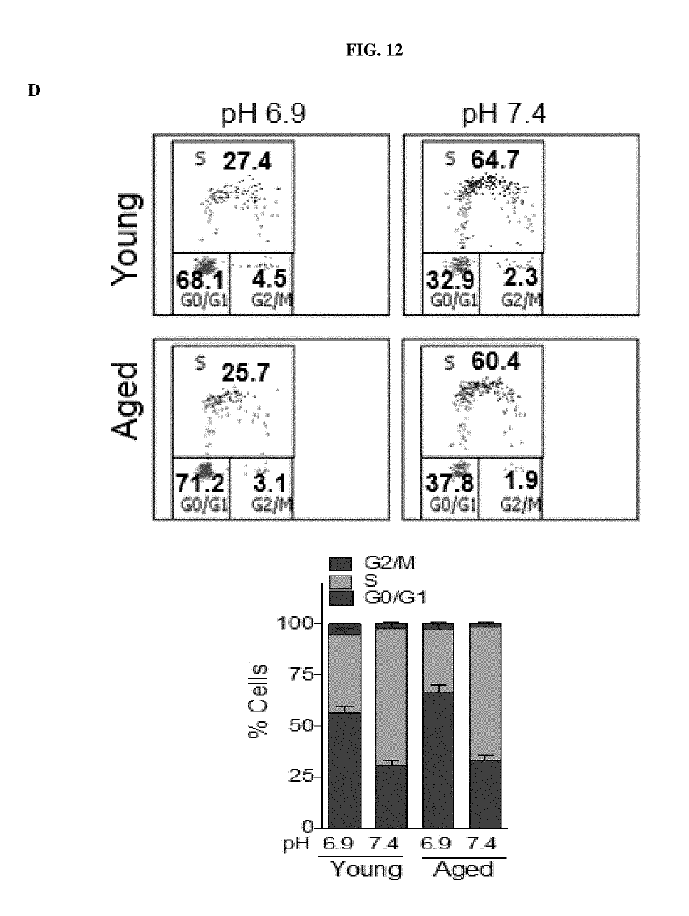

To determine cellular mechanisms linked to the enhanced reconstitution potential of HSCs exposed to a pH of 6.9, we next investigated the effect of pH on HSC cell size (FIG. 11) and proliferation (FIG. 12). At pH 6.9, the size of stem cells was reduced (FIG. 11B). At pH 6.9, both young and aged bone marrow cells presented with an about 2-fold expansion over the number of input cells, while cells at pH 7.4 expanded up to 5-fold (FIG. 12A and FIG. 12B), while the relative expansion of phenotypic stem cells remained similar (FIG. 5). While HSCs entered cell cycle both under pH 6.9 as well as 7.4, there were fewer cells in S phase of the cell division cycle (as determined by EdU incorporation) at pH 6.9 compared to pH 7.4 (FIG. 12C and FIG. 12D). These data suggest that a pH of 6.9 reduces the HSC proliferation rate compared to 7.4. This reduction could imply stem cell function preservation upon expansion.

### Low pH Mitigates Cellular Stress and Regulates Metabolic Targets

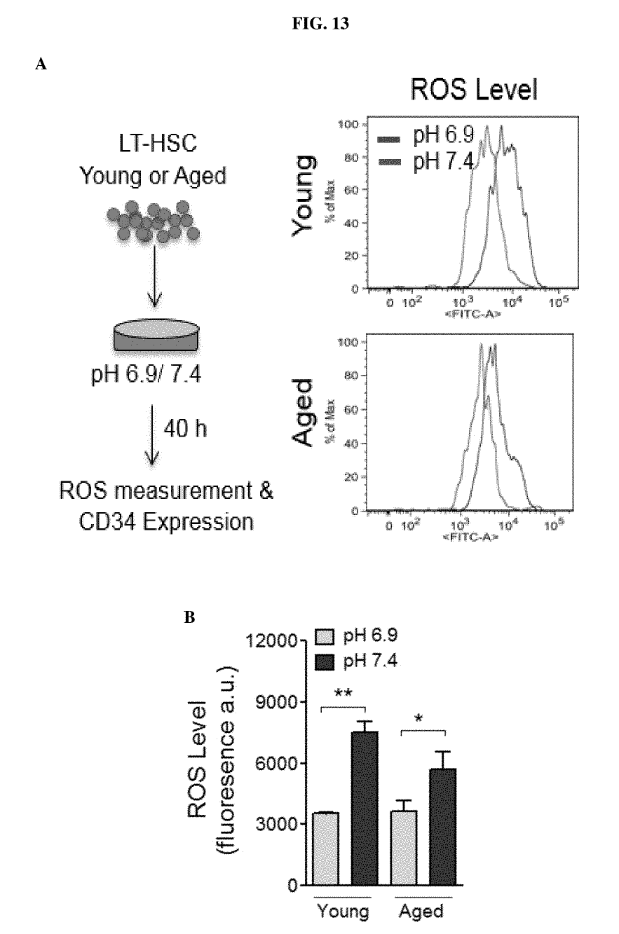

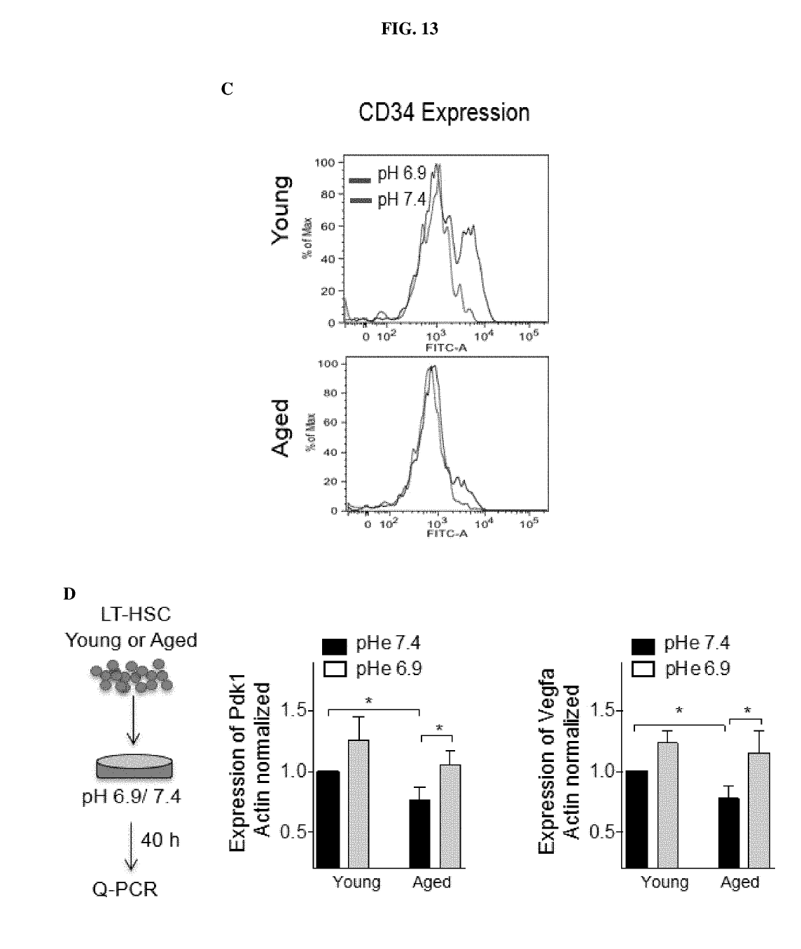

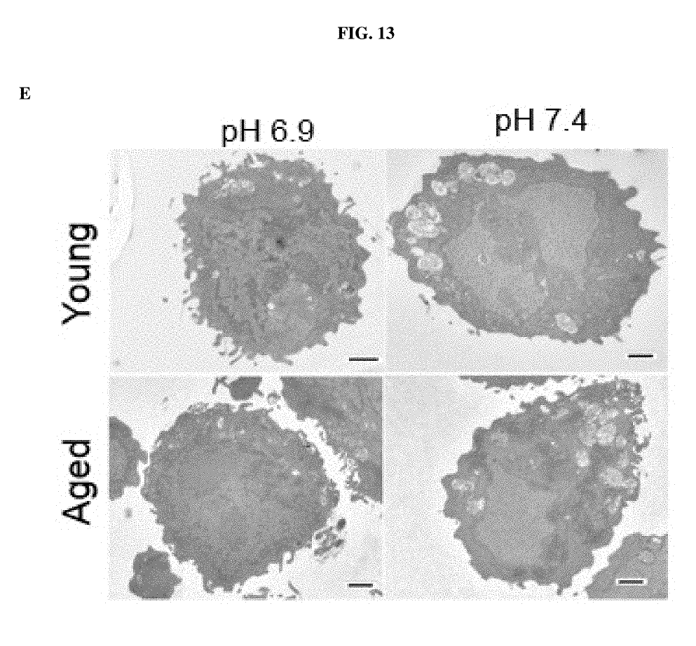

HSCs at pH 6.9 showed less intracellular Reactive Oxygen Species (ROS) (measured via DCF fluorescence) compared to HSC at pH 7.4 in both young and aged HSC, while there appeared to be no difference in ROS in young vs. aged HSCs at the pH 7.4 (FIG. 13A and FIG. 13B). Lack of CD34 expression is a hallmark of HSCs in mice. pH 6.9 exposed HSCs maintained a low-to-zero CD34 level as compared to HSCs at pH 7.4, which induced CD34 expression in 15-30% of HSCs (FIG. 13C), indicating that proliferation at pH 7.4 induces HSC differentiation.

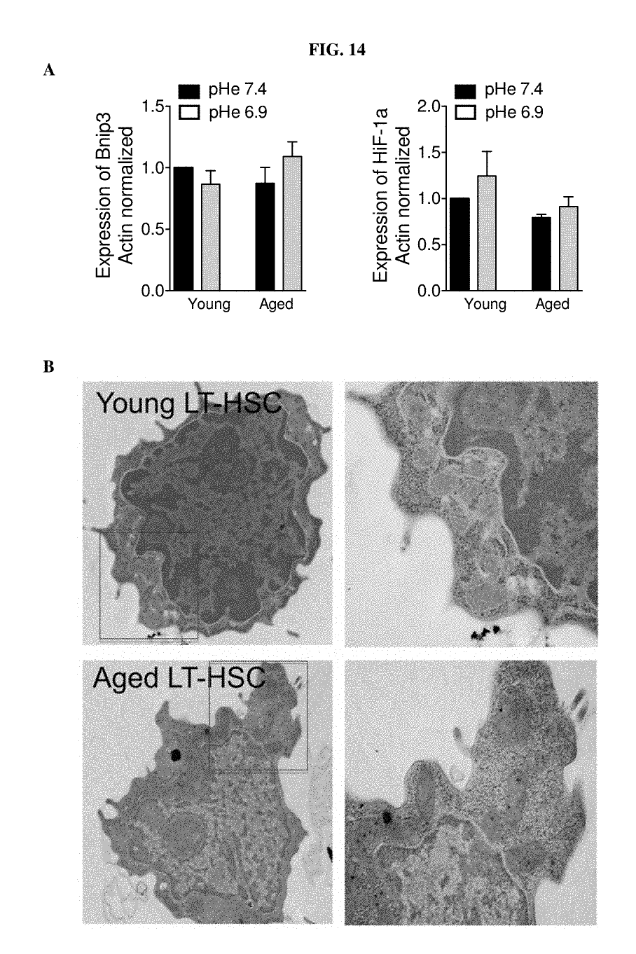

HSCs can exhibit a unique cell metabolism profile. Due to their relative hypoxia, Hif1a is stabilized post-translationally which results in utilization of primarily glycolysis for ATP generation. We next tested whether Hif1a is stabilized in HSCs at a pH 6.9 compared to a pH 7.4. The expression of the Hif1a targets (pdk-1 and vegfa) (FIG. 13D) was increased in pH 6.9 compared to pH 7.4, while Hif-1a expression itself remained mostly unchanged (FIG. 14). This shows that post-translational regulation of Hif1a occurs in HSCs in response to changes in pH.

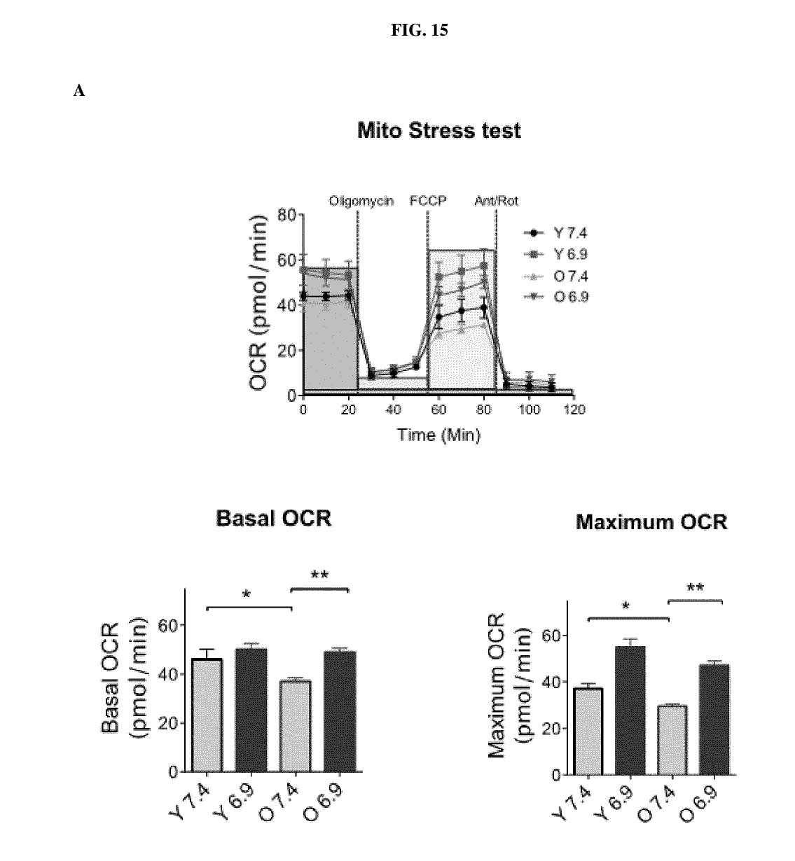

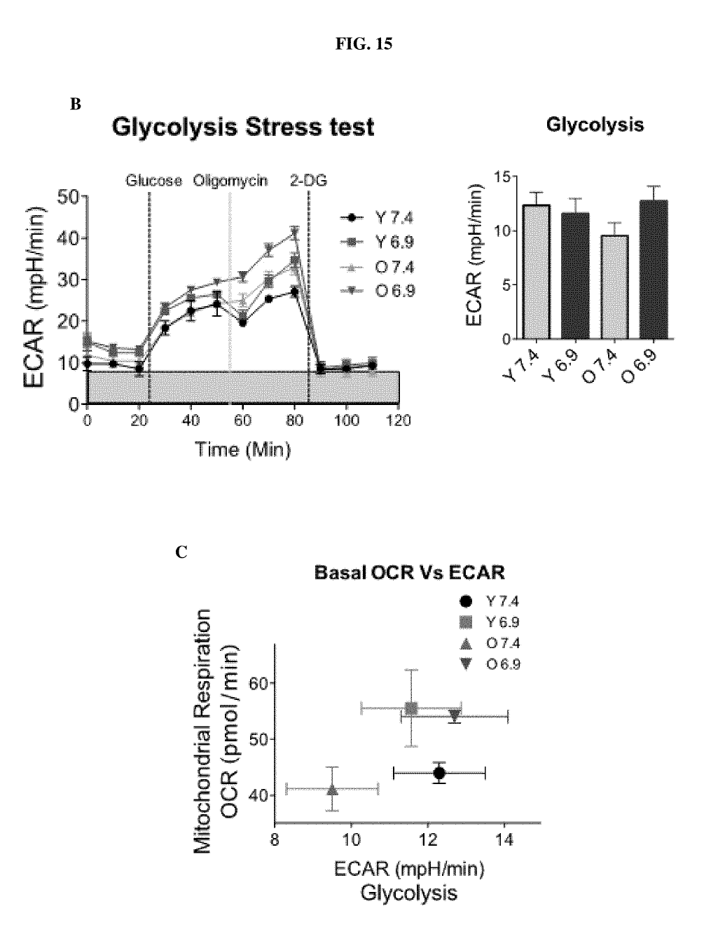

We determined the effect of pH on the structure of the mitochondria in HSCs. Interestingly, we observed elongated and relaxed mitochondria in aged HSCs in contrast to young HSCs that exhibit condensed mitochondria (FIG. 14). pH 6.9 treatment condensed mitochondria in both young and aged HSCs (FIG. 13E), while we observed elongated mitochondria with increased vacuolization under pH 7.4 settings (FIG. 13E); this level of vacuolization is consistent with mitochondrial stress. Mitochondrial respiration parameters analyzed using seahorse technology further implied that pH 6.9 enhances the glycolytic setup of HSCs (FIG. 15).

### Extracellular pH Regulates Intracellular pH, Cytoskeleton Organization and Epipolarity

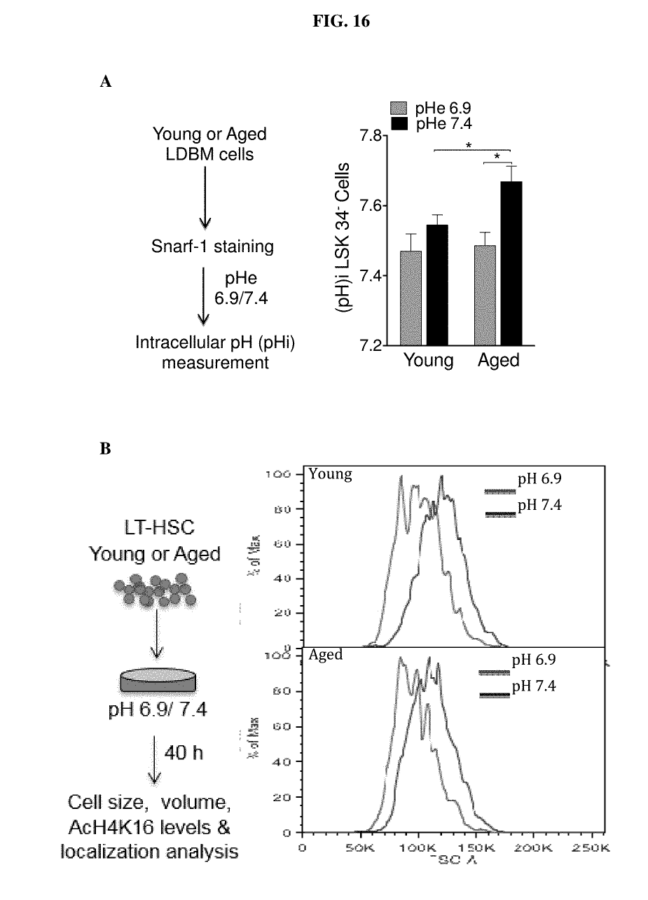

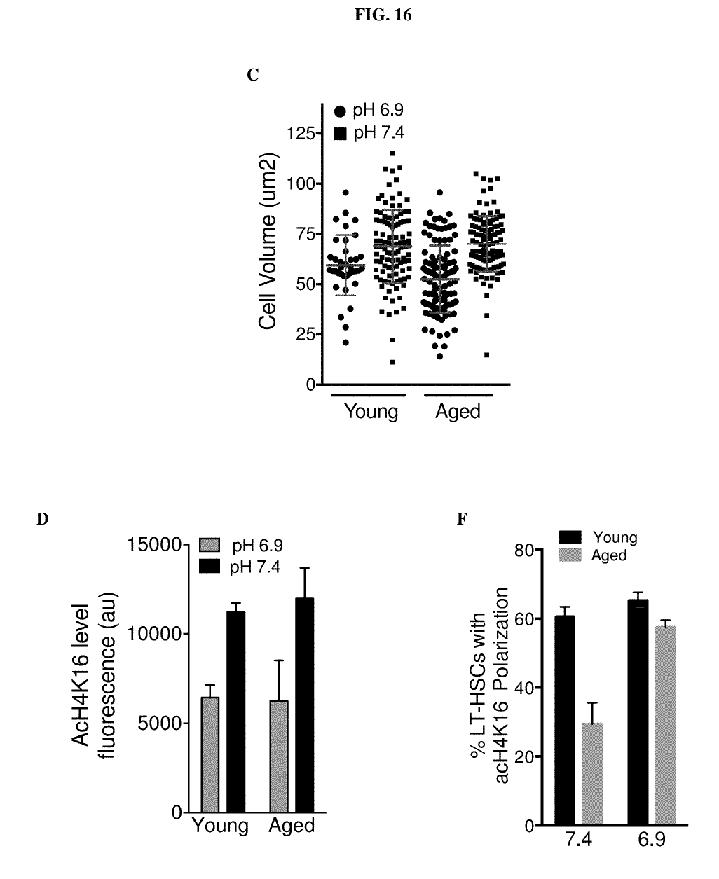

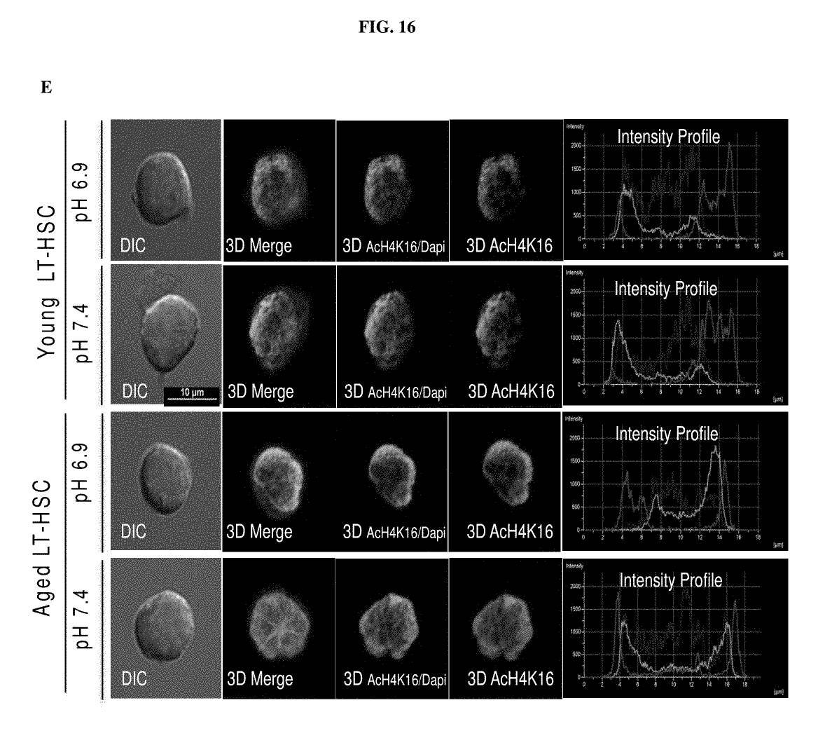

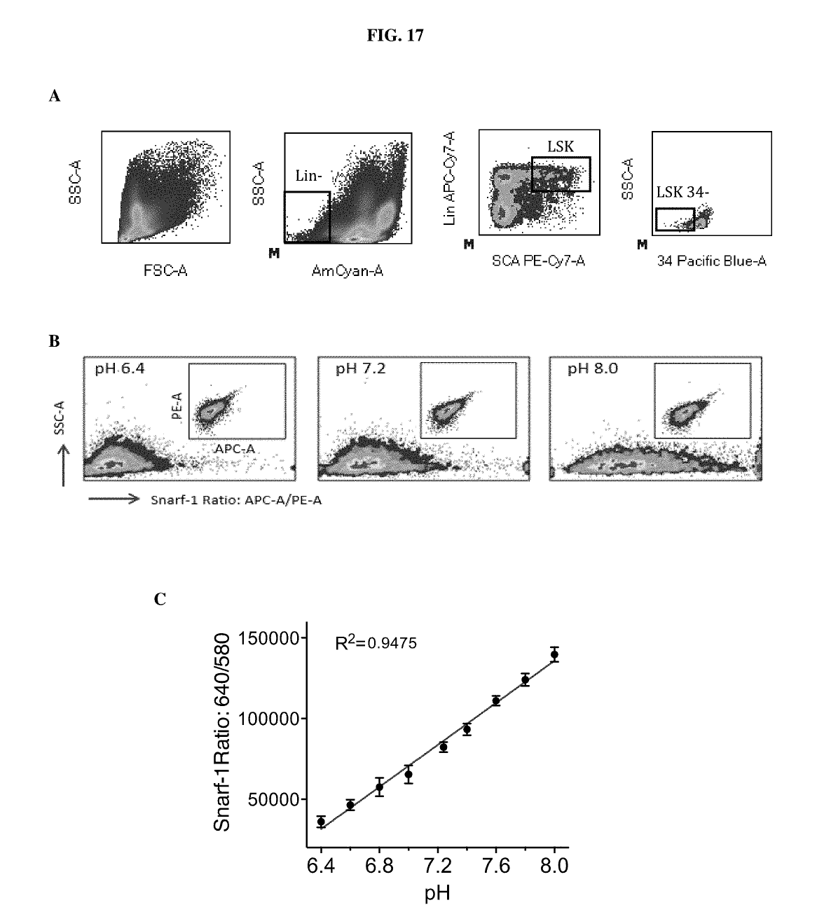

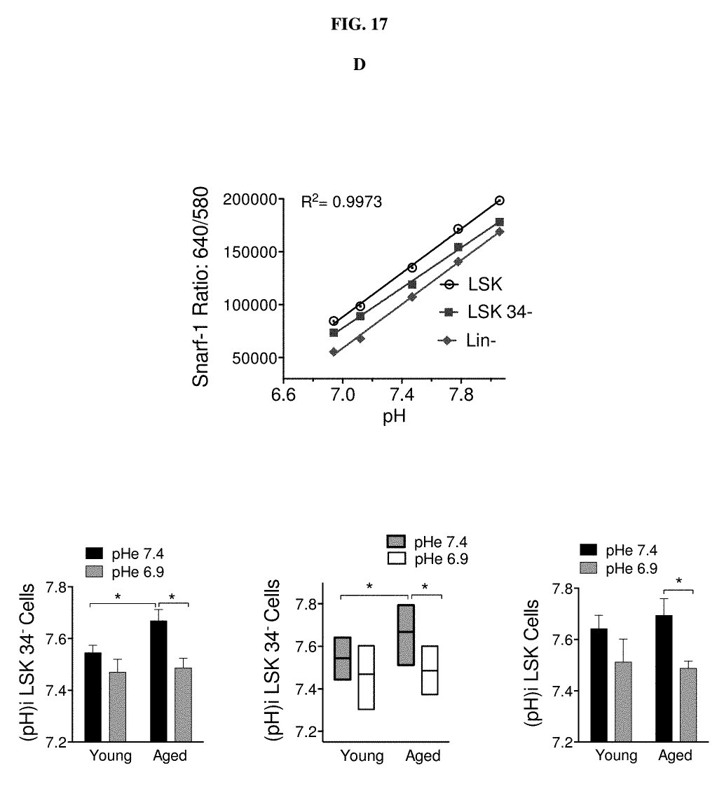

Extracellular pH (pHe) can affect intracellular pH (pHi) in multiple ways. We determined the intracellular pH using carboxy SNARF-1 as a fluorescent intracellular pH probe. pH values of HSCs were calculated based on a pH-SNARF-1 standard curve (FIG. 17C). Aged HSCs, exposed to an extracellular pH of 7.4 for only about 30 mins present with a higher intracellular pH (pHi) compared to young HSCs kept at the same extracellular pH (7.7 to 7.55). A pHe of 6.9 decreased intracellular pH of young HSCs some, but a pHe of 6.9 decreased pHi in aged HSCs to the level reported for young HSCs exposed to a pHe of 6.9 (to about 7.5) (FIG. 16A). A pH of 6.9 decreases the pHi also in more differentiated progenitor cells compared to the pHi when a pHe of 7.4 is applied (FIG. 17 and data not shown). A high intracellular pHi in aged HSCs, which is in contrast to a decrease in BM, seems to counteract mechanisms to maintain better HSC functions in vivo. These data imply that aged HSCs are not as well equipped compared to young HSCs to buffer the extracellular pH at 7.4. They also imply a role of the pHi in regulating some functional changes in HSCs when exposed to a distinct pHe.

Change in cell size as well as volume of HSCs at pHs 6.9 and 7.4 were determined by flow-cytometric and microscopic analyses. Young and aged HSCs presented at pH 6.9 with a decrease in cell size indicated by a decrease in the forward scatter area, which correlates with cell size (FIG. 16B and FIG. 11B) and a decrease in cell area (confocal imaging, FIG. 16C).

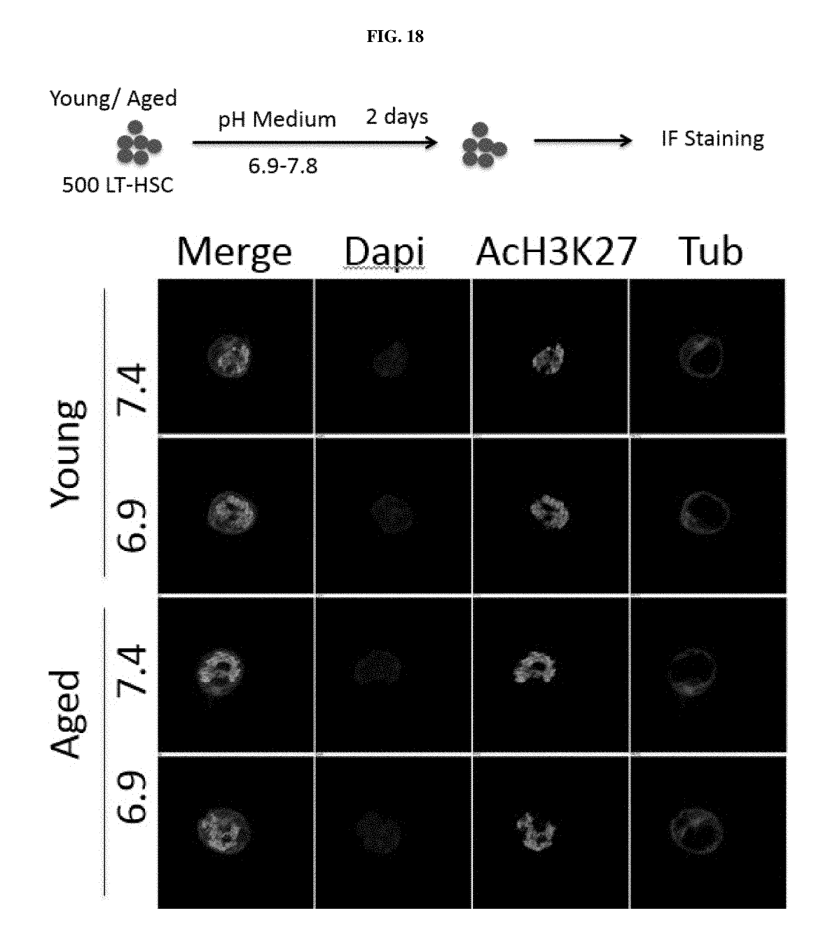

Low pH has been reported to cause a global decrease in HeLa cells and redistribution of the acetylated form of Histone 4 on lysine 16 (H4K16ac) throughout the genome, independently of a specific carbon source or extracellular Na⁺, Cl⁻, Ca²⁺ or phosphate, and it is distinct from changes in the redistribution of H4K16ac in response to nutrient availability. Changes in H4K16ac position within the nucleus of HSCs are also reported to be linked to aging and functional rejuvenation of HSCs. Epigenetic alterations might also be responsible for the “memory” effect of improved function in response to a pH of 6.9 of especially aged HSCs. We thus tested whether changes in pH regulate the distribution of H4K16ac in HSCs. We observed a decrease in the level of H4K16ac at pH 6.9 compared to pH 7.4 in both young and aged HSC using FACS analysis (FIG. 16D), which is consistent with prior reports of a global acetylation decrease at low pH. A specific pattern of the nuclear distribution of H4K16ac (polar) is reported in young HSCs, compared to its apolar nuclear distribution in aged HSCs. A pH 6.9 exposure of aged HSCs increased the frequency of aged HSCs with a polar nuclear localization of H4K16ac to a level similar to that seen in young HSCs while not altering polarity in young HSCs, (FIG. 16E), while no differences in localization and level were observed for H3K27ac (FIG. 18).

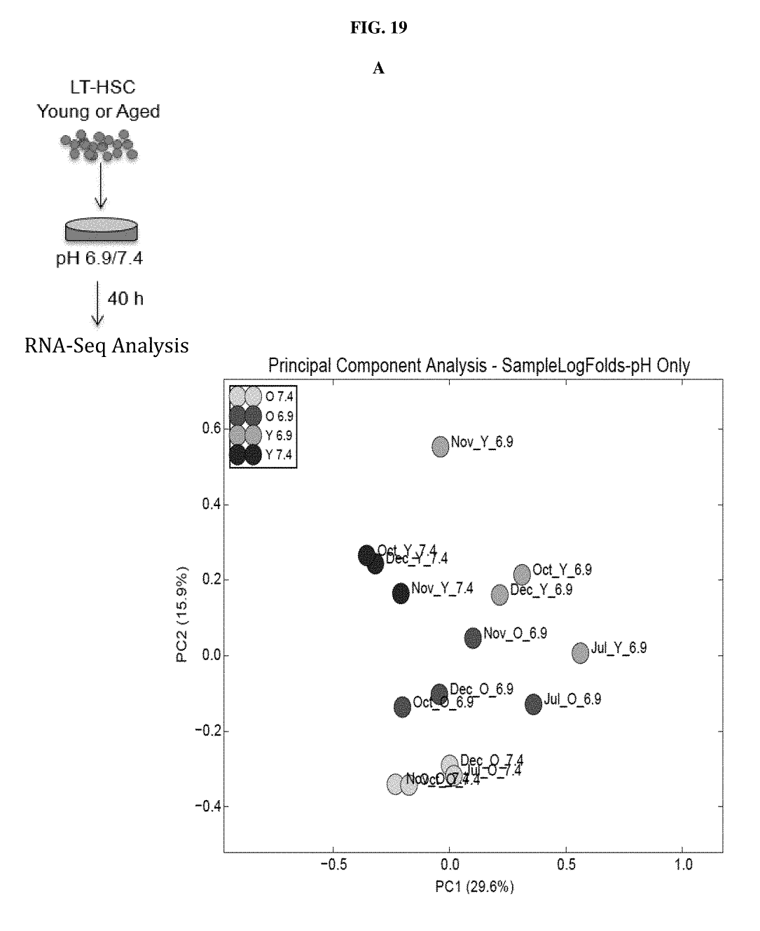

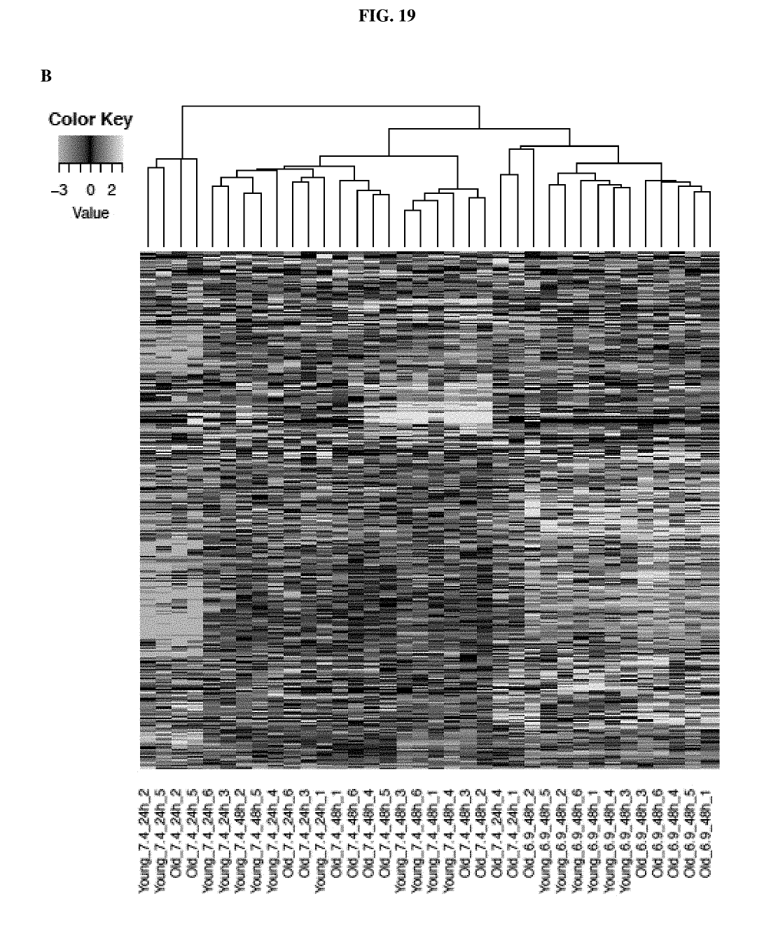

We next performed RNA-seq analysis of young and aged HSC at pH 6.9 or 7.4 (FIG. 19). Principal Component Analysis (PCA) suggests a shift of the overall expression profile of aged HSCs (pH 7.4) towards young HSCs (pH 7.4) after pH 6.9 treatments (FIG. 19A). For more detailed analyses, we assessed the fold change of pH 6.9 vs pH 7.4 in old mice, and then again in young mice to select genes with FC>2 in at least one of those comparisons (FIG. 19B and data not shown). Consistent with functional data, RNA-seq analysis further confirmed low metabolic activity and cellular oxidation at pH 6.9 vs pH 7.4. Glutathione expression increase suggests that HSCs might be metabolically more stable at pH 6.9. Changes in the expression profile of pH 7.4 versus pH 6.9 (both young and aged) treatment correlate with genes controlled by CBFA2T3 (Mtg16), a gene for maintenance of HSCs and which is reported to help control HSC quiescence. This implies that changes in pH confer enhanced quiescence and self-renewal via an CBFA2T3 related mechanism, which might be another link to histone aceylation/de-acetylation reported above, as a role of CBFA2T3 is to link DNA binding transcription factors that control hematopoiesis to chromatin modifying enzymes such as histone deacetylases. In addition to stem known regulatory genes and pathways, genes with the highest negative fold-change were often proteases. Proteases like cathepsin G and granzyme B were down regulated at pH 6.9 treatment, suggesting their expression to be highly regulated/induced by pH 7.4 or suppressed by pH 6.9 in stem cells. This is consistent with a report that human UCB 34+ cells that present higher reconstitution potential than BM CD34+ cells exhibit lower activity of proteases including cathepsin G and elastase. Transcriptome analyses confirm (a) low metabolic active and oxidation state of HSCs at pH 6.9, (b) imply a CBFA2T3 driven pathway for quiescence and expansion upon lower pH and (c) reveal expression of proteases within HSCs as a novel gene category linked to improved HSC function under low pH. Our data thus show that ex vivo low pH 6.9 provides better HSC reconstitution through a decrease in cell proliferation and as a result of low cellular, mitochondrial stress and changes in epigenetics and epi-polarity. Transcriptome analyses suggest that down regulation of proteases as well activation of a CBFA2T3 driven pathway further contribute to a better HSC fitness at pH 6.9 compared to 7.4.

The data reveal extracellular pH as a regulator for stem cell (e.g., murine HSC and human HSC) function and reconstitution ability. A pH of 6.9 in the medium leads to a higher reconstitution ability. An extracellular pH of 6.9 ex vivo also results in at least partial functional rejuvenation of aged murine stem cells (e.g., HSCs). pH regulates stem cell (e.g., HSC) expansion, cellular metabolism and epigenetic polarity. Low cellular oxidation, stabilized metabolism and altered epigenetics induced by a pH of 6.9 are linked to improved stem cell (e.g., HSC) function. These data imply that a pH of 6.9 instead of the standard of 7.4 will support protocols for stem cell (e.g., HSC) maintenance and/or expansion or rejuvenation ex vivo.

The headings used in the disclosure are not meant to suggest that all disclosure relating to the heading is found within the section that starts with that heading. Disclosure for any subject may be found throughout the specification.

It is noted that terms like “preferably,” “commonly,” and “typically” are not used herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

As used in the disclosure, “a” or “an” means one or more than one, unless otherwise specified. As used in the claims, when used in conjunction with the word “comprising” the words “a” or “an” means one or more than one, unless otherwise specified. As used in the disclosure or claims, “another” means at least a second or more, unless otherwise specified. As used in the disclosure, the phrases “such as”, “for example”, and “e.g.” mean “for example, but not limited to” in that the list following the term (“such as”, “for example”, or “e.g.”) provides some examples but the list is not necessarily a fully inclusive list. The word “comprising” means that the items following the word “comprising” may include additional unrecited elements or steps; that is, “comprising” does not exclude additional unrecited steps or elements.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

Detailed descriptions of one or more embodiments are provided herein. It is to be understood, however, that the present invention may be embodied in various forms. Therefore, specific details disclosed herein (even if designated as preferred or advantageous) are not to be interpreted as limiting, but rather are to be used as an illustrative basis for the claims and as a representative basis for teaching one skilled in the art to employ the present invention in any appropriate manner. Indeed, various modifications of the invention in addition to those described herein will become apparent to those skilled in the art from the foregoing description and the accompanying figures. Such modifications are intended to fall within the scope of the appended claims.

What is claimed is:

1. A method for growing first stem cells comprising
growing the first stem cells in a cell medium having a pH of from about 6.6 to about 7.2, and
recovering second stem cells.

2. The method of claim 1, wherein the pH of the cell medium is from about 6.7 to about 7.1.

3. The method of claim 1, wherein the pH of the cell medium is from about 6.8 to about 7.0.

4. The method of claim 1, wherein the pH of the cell medium is about 6.9.

5. The method of claim 1, wherein the first stem cells are grown ex vivo.

6. The method of claim 1, wherein the first stem cells are human stem cells, mouse stem cells, rat stem cells, primate stem cells, or mammalian stem cells.

7. The method of claim 1, wherein the first stem cells are somatic stem cells, tissue-specific stem cells, hematopoietic stem cells (HSC), stem cells that express CD34, mammary stem cells, intestinal stem cells, mesenchymal stem cells, endothelial stem cells, neural stem cells, olfactory adult stem cells, neural crest stem cells, or testicular stem cells.

8. The method of claim 1, wherein the first stem cells are hematopoietic stem cells (HSC) or stem cells that express CD34.

9. The method of claim 1, wherein there is an increase in an amount of chimera cells in peripheral blood, when the second stem cells are competitively transplanted into a recipient, when compared to transplanted stem cells that were grown in cell medium of pH about 7.4.

10. The method of claim 9, where the increase is at least about 20%.

11. The method of claim 1, wherein the second stem cells have a decreased percentage of cells that are in the S phase when compared to stem cells grown in cell medium of pH about 7.4.

12. The method of claim 11, where the decrease is at least about 20%.

13. The method of claim 1, wherein the second stem cells have a decreased amount of reactive oxygen species compared to stem cells grown in cell medium of pH about 7.4.

14. The method of claim 13, where the decrease is at least about 20%.

15. The method of claim 1, wherein the second stem cells have a decreased level of CD34 expression when compared to stem cells grown in cell medium of pH about 7.4.

16. The method of claim 15, where the decrease is at least about 20%.

17. The method of claim 1, wherein the second stem cells have a decreased volume of the stem cell when compared to stem cells grown in cell medium of pH about 7.4.

18. The method of claim 17, where the decrease is at least about 20%.

19. The method of claim 1, wherein the second stem cells have a decreased amount of H4K16ac in the stem cell when compared to stem cells grown in cell medium of pH about 7.4.

20. The method of claim 19, where the decrease is at least about 20%.

21. The method of claim 1, wherein the second stem cells have an increased glutathione expression in the stem cell when compared to stem cells grown in cell medium of pH about 7.4.

22. The method of claim 21, where the increase is at least about 20%.

23. The method of claim 1, wherein the second stem cells have a decreased expression of one or more proteases in the stem cell when compared to stem cells grown in cell medium of pH about 7.4.

24. The method of claim 23, wherein the one or more proteases are cathepsin G, granzyme B, or both.

25. The method of claim 23, where the decrease is at least about 20%.

26. The method of claim 1, wherein the first stem cells are taken from a mouse with an age of at least about 2 months.

27. The method of claim 1, wherein the first stem cells are taken from a mouse with an age of at least about 10 months.

28. The method of claim 1, wherein the first stem cells are taken from a mouse with an age of at least about 18 months.

29. The method of claim 1, wherein the first stem cells are taken from a human with an age of at least about 10 years.

30. The method of claim 1, wherein the first stem cells are taken from a human with an age of at least about 50 years.

31. The method of claim 1, wherein the first stem cells are taken from a human with an age of at least about 65 years.

32. The method of claim 1, wherein the recovering comprises one or more of centrifuging, filtering, or washing.

33. A method for transplanting comprising (a) growing first stem cells in a cell medium having a pH of from about 6.6 to about 7.2, to provide second stem cells and (b) transplanting the second stem cells into a recipient.

34. The method of claim 33, wherein the recipient is a mammal, rodent, primate, monkey, human, canine, feline, porcine, mouse, rabbit, or rat.

35. The method of claim 33, wherein the recipient is a human or mouse.

36. The method of claim 33, wherein the transplanting of the second stem cells in the recipient occurs in the bone, bone marrow, blood, skin, brain, or mammary.

37. The method of claim 33, wherein the pH of the cell medium is from about 6.8 to about 7.0.

38. The method of claim 33, wherein the pH of the cell medium is about 6.9.

39. The method of claim 33, wherein the first stem cells are grown ex vivo.

40. The method of claim 33, wherein the first stem cells are human stem cells, mouse stem cells, rat stem cells, primate stem cells, or mammalian stem cells.

41. The method of claim 33, wherein the first stem cells are somatic stem cells, tissue-specific stem cells, hematopoietic stem cells (HSC), stem cells that express CD34, mammary stem cells, intestinal stem cells, mesenchymal stem cells, endothelial stem cells, neural stem cells, olfactory adult stem cells, neural crest stem cells, or testicular stem cells.

42. The method of claim 33, wherein the first stem cells are hematopoietic stem cells (HSC) or stem cells that express CD34.

43. The method of claim 33, wherein the method further comprises recovering the second stem cells before step (b).
